SEQUENCE LISTING (148 sequences)

SEQ ID NO: 1 - protein, 5 aa, Homo sapiens
NYAIS

SEQ ID NO: 2 - protein, 5 aa, Homo sapiens
SNYIS

SEQ ID NO: 3 - protein, 5 aa, Homo sapiens
NYWIS

SEQ ID NO: 4 - protein, 5 aa, Homo sapiens
SYWIT

SEQ ID NO: 5 - protein, 5 aa, Homo sapiens
NYWIG

SEQ ID NO: 6 - protein, 5 aa, Homo sapiens
SFGMS

SEQ ID NO: 7 - protein, 17 aa, Homo sapiens
GIIPMFGYANYAQKFQG

SEQ ID NO: 8 - protein, 17 aa, Homo sapiens
GIIPVFGFTHYAQKFQG

SEQ ID NO: 9 - protein, 17 aa, Homo sapiens
GIIPIFGHANYAQKFQG

SEQ ID NO: 10 - protein, 17 aa, Homo sapiens
IIIPPIGNAWYAQKFQG

SEQ ID NO: 11 - protein, 17 aa, Homo sapiens
LIDPNFGGAYYAQKFQG

SEQ ID NO: 12 - protein, 17 aa, Homo sapiens
LIDPVFGGAYYAQKFQG

SEQ ID NO: 13 - protein, 17 aa, Homo sapiens
LIDPMFGGAYYAQKFQG

SEQ ID NO: 14 - protein, 16 aa, Homo sapiens
INAHLGGTWYAQKFQG

SEQ ID NO: 15 - protein, 17 aa, Homo sapiens
ISPGTGINAYYAQKFQG

SEQ ID NO: 16 - protein, 17 aa, synthetic construct
  REGION 1..17: note = Synthesized human sequence
  VARIANT 1: note = X = G, I, or L
  VARIANT 2: note = X = I or S
  VARIANT 3: note = X = I, P, N, or D
  VARIANT 4: note = X = P, G, or A
  VARIANT 5: note = X = I, M, P, T, H, N, or V
  VARIANT 6: note = X = F, I, G, or L
  VARIANT 7: note = X = G or I
  VARIANT 8: note = X = H, Y, N, or G
  VARIANT 9: note = X = A or T
  VARIANT 10: note = X = N, W, or Y
XXXXXXXXXXYAQKFQG

SEQ ID NO: 17 - protein, 17 aa, Homo sapiens
WIRPGDSDTRYSPSFEG

SEQ ID NO: 18 - protein, 19 aa, Homo sapiens
VSYISSSGSSTYYADSVKG

SEQ ID NO: 19 - protein, 17 aa, Homo sapiens
IIDPSNSYTNYSPSFQG

SEQ ID NO: 20 - protein, 17 aa, Homo sapiens
IIDPSNSYTRYSPSFQG

SEQ ID NO: 21 - protein, 17 aa, Homo sapiens
IIDPSNSYTDYSPSFQG

SEQ ID NO: 22 - protein, 17 aa, Homo sapiens
IISPTGSVTWYSPSFQG

SEQ ID NO: 23 - protein, 17 aa, Homo sapiens
IISPTGSSTWYSPSFQG

SEQ ID NO: 24 - protein, 17 aa, Homo sapiens
FISPDGSHTWYSPSFQG

SEQ ID NO: 25 - protein, 17 aa, Homo sapiens
IISPSGSTTWYSPSFQG

SEQ ID NO: 26 - protein, 17 aa, Homo sapiens
IISPTGSATWYSPSFQG

SEQ ID NO: 27 - protein, 17 aa, Homo sapiens
IIDPVSSWTKYSPSFQG

SEQ ID NO: 28 - protein, 17 aa, synthetic construct
  REGION 1..17: note = Synthesized human sequence
  VARIANT 3: note = X = D or S
  VARIANT 5: note = X = S, V, D, or T
  VARIANT 6: note = X = N, S, or G
  VARIANT 8: note = X = Y, W, T, H, V, S, or A
  VARIANT 10: note = X = N, D, R, K, or W
IIXPXXSXTXYSPSFQG

SEQ ID NO: 29 - protein, 17 aa, Homo sapiens
NISSSGSSTYYADSVKG

SEQ ID NO: 30 - protein, 19 aa, Homo sapiens
NIEHKYLNYATYYAASVKG

SEQ ID NO: 31 - protein, 19 aa, Homo sapiens
NIEHKFMGYTTYYAAGVKG

SEQ ID NO: 32 - protein, 19 aa, Homo sapiens
GIEHKYLSYTTHYAASVKG

SEQ ID NO: 33 - protein, 19 aa, Homo sapiens
SIEHKYTGYTTYYAAPVKG

SEQ ID NO: 34 - protein, 19 aa, Homo sapiens
QIEHKYLSYTTLYAASVKG

SEQ ID NO: 35 - protein, 19 aa, Homo sapiens
SIEHKYLSYTTFYAASVKG

SEQ ID NO: 36 - protein, 19 aa, Homo sapiens
NIEGKYTSYTTYYAASVKG

SEQ ID NO: 37 - protein, 19 aa, Homo sapiens
GIEHKYLSYATLYAASVKG

SEQ ID NO: 38 - protein, 19 aa, Homo sapiens
NIEHKYLGYATVYAASVKG

SEQ ID NO: 39 - protein, 19 aa, Homo sapiens
SIEHKYLSYATYYAAGVKG

SEQ ID NO: 40 - protein, 8 aa, Homo sapiens
DIYAGMDV

SEQ ID NO: 41 - protein, 18 aa, Homo sapiens
SKKGMYGGWTYPLMMFDL

SEQ ID NO: 42 - protein, 7 aa, Homo sapiens
HYYGMDY

SEQ ID NO: 43 - protein, 12 aa, Homo sapiens
GTFWSFGNYFAN

SEQ ID NO: 44 - protein, 8 aa, Homo sapiens
WYYKPFDV

SEQ ID NO: 45 - protein, 12 aa, Homo sapiens
YWGTPYLMQFDN

SEQ ID NO: 46 - protein, 12 aa, Homo sapiens
RASQSVLGNYLA

SEQ ID NO: 47 - protein, 12 aa, Homo sapiens
RASQSVSSNYLA

SEQ ID NO: 48 - protein, 13 aa, Homo sapiens
SGSSSNIGSYYVN

SEQ ID NO: 49 - protein, 11 aa, Homo sapiens
RASQSIFYNLA

SEQ ID NO: 50 - protein, 14 aa, Homo sapiens
TGSSSNIGSGYDVH

SEQ ID NO: 51 - protein, 14 aa, Homo sapiens
TGTSSDVGGYNSVS

SEQ ID NO: 52 - protein, 7 aa, Homo sapiens
GASSRAT

SEQ ID NO: 53 - protein, 7 aa, Homo sapiens
YASRRAT

SEQ ID NO: 54 - protein, 7 aa, Homo sapiens
GNTHRPS

SEQ ID NO: 55 - protein, 7 aa, Homo sapiens
GASNRAT

SEQ ID NO: 56 - protein, 7 aa, Homo sapiens
GNSKRPS

SEQ ID NO: 57 - protein, 7 aa, Homo sapiens
SVSSRPS

SEQ ID NO: 58 - protein, 9 aa, Homo sapiens
HQYGSISTT

SEQ ID NO: 59 - protein, 9 aa, Homo sapiens
QQYSHLLIT

SEQ ID NO: 60 - protein, 9 aa, Homo sapiens
QQYSHISLT

SEQ ID NO: 61 - protein, 9 aa, Homo sapiens
QQFAHILLT

SEQ ID NO: 62 - protein, 9 aa, Homo sapiens
QQTSNTPFT

SEQ ID NO: 63 - protein, 9 aa, Homo sapiens
QQFITYLPT

SEQ ID NO: 64 - protein, 9 aa, Homo sapiens
QQDALSPFT

SEQ ID NO: 65 - protein, 9 aa, Homo sapiens
QQDRGTPFT

SEQ ID NO: 66 - protein, 9 aa, Homo sapiens
QQSLNIPFT

SEQ ID NO: 67 - protein, 9 aa, Homo sapiens
QQDTSSPFT

SEQ ID NO: 68 - protein, 10 aa, synthetic construct
  REGION 1..10: note = Synthesized human sequence
  VARIANT 3: note = X = T, F, D, or S
  VARIANT 4: note = X = S, I, A, T, R, or L
  VARIANT 5: note = X = N, T, L, S, or G
  VARIANT 6: note = X = T, Y, S, or I
  VARIANT 7: note = X = P or L
  VARIANT 8: note = X = F or P
QQXXXXXXFT

SEQ ID NO: 69 - protein, 11 aa, Homo sapiens
QTYASLGPGEV

SEQ ID NO: 70 - protein, 9 aa, Homo sapiens
QQYSSEPVT

SEQ ID NO: 71 - protein, 9 aa, Homo sapiens
SSWTPSSVV

SEQ ID NO: 72 - protein, 11 aa, Homo sapiens
SSWTDTPNMIV

SEQ ID NO: 73 - protein, 11 aa, Homo sapiens
ASWTDGLSLVV

SEQ ID NO: 74 - protein, 11 aa, synthetic construct
  REGION 1..11: note = Synthesized human sequence
  VARIANT 1: note = X = S or A
  VARIANT 6: note = X = T or G
  VARIANT 7: note = X = P or L
  VARIANT 8: note = X = S or N
  VARIANT 9: note = X = S, M, or L
  VARIANT 10: note = X = I or V
XSWTDXXXXXV

SEQ ID NO: 75 - protein, 11 aa, Homo sapiens
SSYDTNKPLVV

SEQ ID NO: 76 - protein, 11 aa, Homo sapiens
GSYDVYGRFYV

SEQ ID NO: 77 - protein, 11 aa, Homo sapiens
SSYYFYLQRIV

SEQ ID NO: 78 - protein, 11 aa, Homo sapiens
QTYYFSYSGPV

SEQ ID NO: 79 - protein, 11 aa, Homo sapiens
GSWDPIFSYEV

SEQ ID NO: 80 - protein, 117 aa, Homo sapiens
QVQLVQSGAEVKKPGSSVKVSCKASGGTFSNYAISWVRQAPGQGLEWMGGIIPMFGYANYAQKFQGRVTITADESTSTAYMELSSLRSEDTAVYYCARDIYAGMDVWGQGTLVTVSS

SEQ ID NO: 81 - protein, 117 aa, Homo sapiens
QVQLVQSGAEVKKPGSSVKVSCKASGGTFSNYAISWVRQAPGQGLEWMGGIIPVFGFTHYAQKFQGRVTITADESTSTAYMELSSLRSEDTAVYYCARDIYAGMDVWGQGTLVTVSS

SEQ ID NO: 82 - protein, 108 aa, Homo sapiens
DIVLTQSPATLSLSPGERATLSCRASQSVLGNYLAWYQQKPGQAPRLLIYGASSRATGVPARFSGSGSGTDFTLTISSLEPEDFAVYYCHQYGSISTTFGQGTKVEIK

SEQ ID NO: 83 - protein, 108 aa, Homo sapiens
DIVLTQSPATLSLSPGERATLSCRASQSVLGNYLAWYQQKPGQAPRLLIYGASSRATGVPARFSGSGSGTDFTLTISSLEPEDFAVYYCQQYSHISLTFGQGTKVEIK

SEQ ID NO: 84 - protein, 108 aa, Homo sapiens
DIVLTQSPATLSLSPGERATLSCRASQSVLGNYLAWYQQKPGQAPRLLIYGASSRATGVPARFSGSGSGTDFTLTISSLEPEDFAVYYCQQYSHLIITFGQGTKVEIK

SEQ ID NO: 85 - protein, 108 aa, Homo sapiens
DIVLTQSPATLSLSPGERATLSCRASQSVLGNYLAWYQQKPGQAPRLLIYGASSRATGVPARFSGSGSGTDFTLTISSLEPEDFAVYYCQQFAHILLTFGQGTKVEIK

SEQ ID NO: 86 - protein, 127 aa, Homo sapiens
QVQLVQSGAEVKKPGSSVKVSCKASGGTFSSNYISWVRQAPGQGLEWMGGIIPIFGHANYAQKFQGRVTITADESTSTAYMELSSLRSEDTAVYYCARSKKGMYGGWTYPLMMFDLWGQGTLVTVSS

SEQ ID NO: 87 - protein, 127 aa, Homo sapiens
QVQLVQSGAEVKKPGSSVKVSCKASGGTFSSNYISWVRQAPGQGLEWMGIIIPPIGNAWYAQKFQGRVTITADESTSTAYMELSSLRSEDTAVYYCARSKKGMYGGWTYPLMMFDLWGQGTLVTVSS

SEQ ID NO: 88 - protein, 127 aa, Homo sapiens
QVQLVQSGAEVKKPGSSVKVSCKASGGTFSSNYISWVRQAPGQGLEWMGISPGTGINAYYAQKFQGRVTITADESTSTAYMELSSLRSEDTAVYYCARSKKGMYGGWTYPLMMFDLWGQGTLVTVSS

SEQ ID NO: 89 - protein, 126 aa, Homo sapiens
QVQLVQSGAEVKKPGSSVKVSCKASGGTFSSNYISWVRQAPGQGLEWMGINAHLGGTWYAQKFQGRVTITADESTSTAYMELSSLRSEDTAVYYCARSKKGMYGGWTYPLMMFDLWGQGTLVTVSS

SEQ ID NO: 90 - protein, 127 aa, Homo sapiens
QVQLVQSGAEVKKPGSSVKVSCKASGGTFSSNYISWVRQAPGQGLEWMGLIDPNFGGAYYAQKFQGRVTITADESTSTAYMELSSLRSEDTAVYYCARSKKGMYGGWTYPLMMFDLWGQGTLVTVSS

SEQ ID NO: 91 - protein, 127 aa, Homo sapiens
QVQLVQSGAEVKKPGSSVKVSCKASGGTFSSNYISWVRQAPGQGLEWMGLIDPVFGGAYYAQKFQGRVTITADESTSTAYMELSSLRSEDTAVYYCARSKKGMYGGWTYPLMMFDLWGQGTLVTVSS

SEQ ID NO: 92 - protein, 127 aa, Homo sapiens
QVQLVQSGAEVKKPGSSVKVSCKASGGTFSSNYISWVRQAPGQGLEWMGLIDPMFGGAYYAQKFQGRVTITADESTSTAYMELSSLRSEDTAVYYCARSKKGMYGGWTYPLMMFDLWGQGTLVTVSS

SEQ ID NO: 93 - protein, 108 aa, Homo sapiens
DIVLTQSPATLSLSPGERATLSCRASQSVSSNYLAWYQQKPGQAPRLLIYYASRRATGVPARFSGSGSGTDFTLTISSLEPEDFATYYCQQTSNTPFTFGQGTKVEIK

SEQ ID NO: 94 - protein, 108 aa, Homo sapiens
EIVLTQSPATLSLSPGERATLSCRASQSVSSNYLAWYQQKPGQAPRLLIYYASRRATGVPARFSGSGSGTDFTLTISSLEPEDFAVYYCQQTSNTPFTFGQGTKVEIK

SEQ ID NO: 95 - protein, 108 aa, Homo sapiens
DIVLTQSPATLSLSPGERATLSCRASQSVSSNYLAWYQQKPGQAPRLLIYYASRRATGVPARFSGSGSGTDFTLTISSLEPEDFAVYYCQQFITYLPTFGQGTKVEIK

SEQ ID NO: 96 - protein, 108 aa, Homo sapiens
DIVLTQSPATLSLSPGERATLSCRASQSVSSNYLAWYQQKPGQAPRLLIYYASRRATGVPARFSGSGSGTDFTLTISSLEPEDFAVYYCQQDALSPFTFGQGTKVEIK

SEQ ID NO: 97 - protein, 108 aa, Homo sapiens
DIVLTQSPATLSLSPGERATLSCRASQSVSSNYLAWYQQKPGQAPRLLIYYASRRATGVPARFSGSGSGTDFTLTISSLEPEDFAVYYCQQDRGTPFTFGQGTKVEIK

SEQ ID NO: 98 - protein, 108 aa, Homo sapiens
DIVLTQSPATLSLSPGERATLSCRASQSVSSNYLAWYQQKPGQAPRLLIYYASRRATGVPARFSGSGSGTDFTLTISSLEPEDFAVYYCQQSLNIPFTFGQGTKVEIK

SEQ ID NO: 99 - protein, 116 aa, Homo sapiens
QVQLVQSGAEVKKPGESLKISCKGSGYSFTNYWISWVRQAPGKGLEWMGWIRPGDSDTRYSPSFEGQVTISADKSISTAYLQWSSLKASDTAMYYCARHYYGMDYWGQGTLVTVSS

SEQ ID NO: 100 - protein, 110 aa, Homo sapiens
DIVLTQPPSVSGAPGQRVTISCSGSSSNIGSYYVNWYQQLPGTAPKLLIYGNTHRPSGVPDRFSGSKSGTSASLAITGLQSEDEADYYCQTYASLGPGEVFGGGTKLTVL

SEQ ID NO: 101 - protein, 121 aa, Homo sapiens
QVQLVESGGGLVQPGGSLRLSCAASGFTFSSYWITWVRQAPGKGLEWVSYISSSGSSTYYADSVKGRFTISRDNSKNTLYLQMNSLRAEDTAVYYCARGTFWSFGNYFANWGQGTLVTVSS

SEQ ID NO: 102 - protein, 107 aa, Homo sapiens
DIVLTQSPATLSLSPGERATLSCRASQSIFYNLAWYQQKPGQAPRLLIYGASNRATGVPARFSGSGSGTDFTLTISSLEPEDFATYYCQQYSSEPVTFGQGTKVEIK

SEQ ID NO: 103 - protein, 117 aa, Homo sapiens
QVQLVQSGAEVKKPGESLKISCKGSGYSFSNYWIGWVRQMPGKGLEWMGIIDPSNSYTNYSPSFQGQVTISADKSISTAYLQWSSLKASDTAMYYCARWYYKPFDVWGQGTLVTVSS

SEQ ID NO: 104 - protein, 117 aa, Homo sapiens
QVQLVQSGAEVKKPGESLKISCKGSGYSFSNYWIGWVRQMPGKGLEWMGIIDPSNSYTDYSPSFQGQVTISADKSISTAYLQWSSLKASDTAMYYCARWYYKPFDVWGQGTLVTVSS

SEQ ID NO: 105 - protein, 117 aa, Homo sapiens
QVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 106 - protein, 117 aa, Homo sapiens
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 107 - protein, 117 aa, Homo sapiens
QVQLVQSGAEVKKPGESLKISCKGSGYSFSNYWIGWVRQMPGKGLEWMGIIDPVSSWTKYSPSFQGQVTISADKSISTAYLQWSSLKASDTAMYYCARWYYKPFDVWGQGTLVTVSS

SEQ ID NO: 108 - protein, 117 aa, Homo sapiens
QVQLVQSGAEVKKPGESLKISCKGSGYSFSNYWIGWVRQMPGKGLEWMGIISPSGSTTWYSPSFQGQVTISADKSISTAYLQWSSLKASDTAMYYCARWYYKPFDVWGQGTLVTVSS

SEQ ID NO: 109 - protein, 117 aa, Homo sapiens
QVQLVQSGAEVKKPGESLKISCKGSGYSFSNYWIGWVRQMPGKGLEWMGFISPDGSHTWYSPSFQGQVTISADKSISTAYLQWSSLKASDTAMYYCARWYYKPFDVWGQGTLVTVSS

SEQ ID NO: 110 - protein, 117 aa, Homo sapiens
QVQLVQSGAEVKKPGESLKISCKGSGYSFSNYWIGWVRQMPGKGLEWMGIISPTGSVTWYSPSFQGQVTISADKSISTAYLQWSSLKASDTAMYYCARWYYKPFDVWGQGTLVTVSS

SEQ ID NO: 111 - protein, 117 aa, Homo sapiens
QVQLVQSGAEVKKPGESLKISCKGSGYSFSNYWIGWVRQMPGKGLEWMGIISPTGSSTWYSPSFQGQVTISADKSISTAYLQWSSLKASDTAMYYCARWYYKPFDVWGQGTLVTVSS

SEQ ID NO: 112 - protein, 117 aa, Homo sapiens
QVQLVQSGAEVKKPGESLKISCKGSGYSFSNYWIGWVRQMPGKGLEWMGIISPTGSATWYSPSFQGQVTISADKSISTAYLQWSSLKASDTAMYYCARWYYKPFDVWGQGTLVTVSS

SEQ ID NO: 113 - protein, 109 aa, Homo sapiens
DIVLTQPPSVSGAPGQRVTISCTGSSSNIGSGYDVHWYQQLPGTAPKLLIYGNSKRPSGVPDRFSGSKSGTSASLAITGLQSEDEADYYCSSWTPSSVVFGGGTKLTVL

SEQ ID NO: 114 - protein, 111 aa, Homo sapiens
DIVLTQPPSVSGAPGQRVTISCTGSSSNIGSGYDVHWYQQLPGTAPKLLIYGNSKRPSGVPDRFSGSKSGTSASLAITGLQSEDEADYYCSSWTDTPNMIVFGGGTKLTVL

SEQ ID NO: 115 - protein, 111 aa, Homo sapiens
DIVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 116 - protein, 111 aa, Homo sapiens
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 117 - protein, 121 aa, Homo sapiens
QVQLVESGGGLVQPGGSLRLSCAASGFTFSSFGMSWVRQAPGKGLEWVSNISSSGSSTYYADSVKGRFTISRDNSKNTLYLQMNSLRAEDTAVYYCARYWGTPYLMQFDNWGQGTLVTVSS

SEQ ID NO: 118 - protein, 123 aa, Homo sapiens
QVQLVESGGGLVQPGGSLRLSCAASGFTFSSFGMSWVRQAPGKGLEWVSNIEHKFMGYTTYYAAGVKGRFTISRDNSKNTLYLQMNSLRAEDTAVYYCARYWGTPYLMQFDNWGQGTLVTVSS

SEQ ID NO: 119 - protein, 123 aa, Homo sapiens
QVQLVESGGGLVQPGGSLRLSCAASGFTFSSFGMSWVRQAPGKGLEWVSSIEHKYTGYTTYYAAPVKGRFTISRDNSKNTLYLQMNSLRAEDTAVYYCARYWGTPYLMQFDNWGQGTLVTVSS

SEQ ID NO: 120 - protein, 123 aa, Homo sapiens
QVQLVESGGGLVQPGGSLRLSCAASGFTFSSFGMSWVRQAPGKGLEWVSNIEHKYTSYTTYYAASVKGRFTISRDNSKNTLYLQMNSLRAEDTAVYYCARYWGTPYLMQFDNWGQGTLVTVSS

SEQ ID NO: 121 - protein, 123 aa, Homo sapiens
QVQLVESGGGLVQPGGSLRLSCAASGFTFSSFGMSWVRQAPGKGLEWVSNIEHKYLNYATYYAASVKGRFTISRDNSKNTLYLQMNSLRAEDTAVYYCARYWGTPYLMQFDNWGQGTLVTVSS

SEQ ID NO: 122 - protein, 123 aa, Homo sapiens
QVQLVESGGGLVQPGGSLRLSCAASGFTFSSFGMSWVRQAPGKGLEWVSNIEHKYLGYATVYAASVKGRFTISRDNSKNTLYLQMNSLRAEDTAVYYCARYWGTPYLMQFDNWGQGTLVTVSS

SEQ ID NO: 123 - protein, 123 aa, Homo sapiens
QVQLVESGGGLVQPGGSLRLSCAASGFTFSSFGMSWVRQAPGKGLEWVSSIEHKYLSYATYYAAGVKGRFTISRDNSKNTLYLQMNSLRAEDTAVYYCARYWGTPYLMQFDNWGQGTLVTVSS

SEQ ID NO: 124 - protein, 123 aa, Homo sapiens
QVQLVESGGGLVQPGGSLRLSCAASGFTFSSFGMSWVRQAPGKGLEWVSSIEHKYLSYTTFYAASVKGRFTISRDNSKNTLYLQMNSLRAEDTAVYYCARYWGTPYLMQFDNWGQGTLVTVSS

SEQ ID NO: 125 - protein, 123 aa, Homo sapiens
QVQLVESGGGLVQPGGSLRLSCAASGFTFSSFGMSWVRQAPGKGLEWVSGIEHKYLSYTTHYAASVKGRFTISRDNSKNTLYLQMNSLRAEDTAVYYCARYWGTPYLMQFDNWGQGTLVTVSS

SEQ ID NO: 126 - protein, 123 aa, Homo sapiens
QVQLVESGGGLVQPGGSLRLSCAASGFTFSSFGMSWVRQAPGKGLEWVSQIEHKYLSYTTLYAASVKGRFTISRDNSKNTLYLQMNSLRAEDTAVYYCARYWGTPYLMQFDNWGQGTLVTVSS

SEQ ID NO: 127 - protein, 123 aa, Homo sapiens
QVQLVESGGGLVQPGGSLRLSCAASGFTFSSFGMSWVRQAPGKGLEWVSGIEHKYLSYATLYAASVKGRFTISRDNSKNTLYLQMNSLRAEDTAVYYCARYWGTPYLMQFDNWGQGTLVTVSS

SEQ ID NO: 128 - protein, 111 aa, Homo sapiens
DIALTQPASVSGSPGQSITISCTGTSSDVGGYNSVSWYQQHPGKAPKLMIYSVSSRPSGVSNRFSGSKSGNTASLTISGLQAEDEADYYCSSYDTNKPLVVFGGGTKLTVL

SEQ ID NO: 129 - protein, 111 aa, Homo sapiens
DIALTQPASVSGSPGQSITISCTGTSSDVGGYNSVSWYQQHPGKAPKLMIYSVSSRPSGVSNRFSGSKSGNTASLTISGLQAEDEADYYCSSYYFYLQRIVFGGGTKLTVL

SEQ ID NO: 130 - protein, 111 aa, Homo sapiens
DIALTQPASVSGSPGQSITISCTGTSSDVGGYNSVSWYQQHPGKAPKLMIYSVSSRPSGVSNRFSGSKSGNTASLTISGLQAEDEADYYCQTYYFSYSGPVFGGGTKLTVL

SEQ ID NO: 131 - protein, 111 aa, Homo sapiens
DIALTQPASVSGSPGQSITISCTGTSSDVGGYNSVSWYQQHPGKAPKLMIYSVSSRPSGVSNRFSGSKSGNTASLTISGLQAEDEADYYCGSYDVYGRFYVFGGGTKLTVL

SEQ ID NO: 132 - protein, 111 aa, Homo sapiens
DIALTQPASVSGSPGQSITISCTGTSSDVGGYNSVSWYQQHPGKAPKLMIYSVSSRPSGVSNRFSGSKSGNTASLTISGLQAEDEADYYCGSWDPIFSYEVFGGGTKLTVL

SEQ ID NO: 133 - DNA, 381 bp, Homo sapiens
  source 1..381: mol_type = unassigned DNA
caggtgcagctggtgcagtctggggctgaggtgaagaagcctgggtcctcggtgaaggtctcctgcaaggcttctggaggcaccttcagcagcaactacatcagctgggtgcgacaggcccctggacaagggcttgagtggatggggatcagccctggcaccggtatcaacgcatactacgcacagaagttccagggcagagtcacgattaccgcggacgaatccacgagcacagcctacatggagctgagcagcctgagatctgaggacacggccgtgtattactgtgcgagaagcaagaagggcatgtacggcggctggacctaccccctgatgatgttcgacctgtggggccagggcaccctggtgaccgtgagcagc

SEQ ID NO: 134 - DNA, 381 bp, Homo sapiens
  source 1..381: mol_type = unassigned DNA
caggtgcagctggtgcagagcggcgccgaggtgaagaagcccggcagcagcgtgaaggtgagctgcaaggccagcggcggcaccttcagcagcaactacatcagctgggtgcgccaggcccccggccagggcctggagtggatgggcatcagccccggcaccggcatcaacgcctactacgcccagaagttccagggccgcgtgaccatcaccgccgacgagagcaccagcaccgcctacatggagctgagcagcctgcgcagcgaggacaccgccgtgtactactgcgcccgcagcaagaagggcatgtacggcggctggacctaccccctgatgatgttcgacctgtggggccagggcaccctggtgaccgtgagcagc

SEQ ID NO: 135 - DNA, 381 bp, Homo sapiens
  source 1..381: mol_type = unassigned DNA
caggtgcaattggttcagtctggcgcggaagtgaaaaaaccgggcagcagcgtgaaagtgagctgcaaagcctccggaggcactttttcttctaattatatttcttgggtgcgccaagcccctgggcagggtctcgagtggatgggcatttctcctggtactggtattaatgcttattatgctcagaagtttcagggtcgggtgaccattaccgcggatgaaagcaccagcaccgcgtatatggaactgagcagcctgcgtagcgaagatacggccgtgtattattgcgcgcgttctaagaagggtatgtatggtggttggacttatcctcttatgatgtttgatctttggggccaaggcaccctggtgacggttagctca

SEQ ID NO: 136 - DNA, 324 bp, Homo sapiens
  source 1..324: mol_type = unassigned DNA
gagatcgtgctgacccagagccccgccaccctgagcctgagccccggcgagcgcgccaccctgagctgccgcgccagccagagcgtgagcagcaactacctggcctggtaccagcagaagcccggccaggccccccgcctgctgatctactacgccagccgccgcgccaccggcgtgcccgcccgcttcagcggcagcggcagcggcaccgacttcaccctgaccatcagcagcctggagcccgaggacttcgccgtgtactactgccagcagaccagcaacacccccttcaccttcggccagggcaccaaggtggagatcaag

SEQ ID NO: 137 - DNA, 324 bp, Homo sapiens
  source 1..324: mol_type = unassigned DNA
gaaattgtgttgacacagtctccagccaccctgtctttgtctccaggggaaagagccaccctctcctgcagggccagtcagagtgttagcagcaactacttagcctggtaccaacagaaacctggccaggctcccaggctcctcatctattacgcatcccgcagggccactggcgtgccagccaggttcagtggcagtgggtctgggacagacttcactctcaccatcagcagcctagagcctgaagattttgcagtttattactgtcagcagacttctaatactccttttacctttggccagggtacgaaagttgaaattaaa

SEQ ID NO: 138 - DNA, 324 bp, Homo sapiens
  source 1..324: mol_type = unassigned DNA
gagatcgtgctgacccagagcccggcgaccctgagcctgtctccgggcgaacgtgcgaccctgagctgcagagcgagccagtctgtttcttctaattatctggcttggtaccagcagaaaccaggtcaagcaccgcgtctattaatttattatgcttctcgtcgtgcaactggggtcccggcgcgttttagcggctctggatccggcacggattttaccctgaccattagcagcctggaacctgaagactttgcggtgtattattgccagcagacttctaatactccttttacctttggccagggtacgaaagttgaaattaaa

SEQ ID NO: 139 - DNA, 351 bp, Homo sapiens
  source 1..351: mol_type = unassigned DNA
gaggtgcagctggtgcagtctggagcagaggtgaaaaagcccggggagtctctgaagatctcctgtaagggttctggatacagctttagcaactactggatcggctgggtgcgccagatgcccgggaaaggcctggagtggatggggatcatcgaccctagcaactcttacaccagatacagcccgtccttccaaggccaggtcaccatctcagccgacaagtccatcagcaccgcctacctgcagtggagcagcctgaaggcctcggacaccgccatgtattactgtgcgagatggtactacaagcccttcgacgtgtggggccagggcaccctggtgaccgtgagcagc

SEQ ID NO: 140 - DNA, 351 bp, Homo sapiens
  source 1..351: mol_type = unassigned DNA
gaggtgcagctggtgcagagcggcgccgaggtgaagaagcccggcgagagcctgaagatcagctgcaagggcagcggctacagcttcagcaactactggatcggctgggtgcgccagatgcccggcaagggcctggagtggatgggcatcatcgaccccagcaacagctacacccgctacagccccagcttccagggccaggtgaccatcagcgccgacaagagcatcagcaccgcctacctgcagtggagcagcctgaaggccagcgacaccgccatgtactactgcgcccgctggtactacaagcccttcgacgtgtggggccagggcaccctggtgaccgtgagcagc

SEQ ID NO: 141 - DNA, 351 bp, Homo sapiens
  source 1..351: mol_type = unassigned DNA
gaggtgcaattggttcagagcggcgcggaagtgaaaaaaccgggcgaaagcctgaaaattagctgcaaaggttccggatattccttttctaattattggattggttgggtgcgccagatgcctgggaagggtctcgagtggatgggcattatcgatccgtctaatagctatacccgctattctccgagctttcagggccaggtgaccattagcgcggataaaagcattagcaccgcgtatcttcaatggagcagcctgaaagcgagcgatacggccatgtattattgcgcgcgttggtattataagccttttgatgtttggggccaaggcaccctggtgacggttagctca

SEQ ID NO: 142 - DNA, 336 bp, Homo sapiens
  source 1..336: mol_type = unassigned DNA
cagtctgtgctgacgcagccgccctcagtgtctggggccccagggcagagggtcaccatctcctgcactgggagcagctccaacatcgggagcggttatgatgtacactggtaccagcagcttccaggaacagcccccaaactcctcatctatggtaacagcaagcggccctcaggggtccctgaccgattctctggctccaagtctggcacctcagcctccctggccatcactgggctccagagcgaggatgaggctgattattactgcgccagctggaccgacggcctgagcctggtggtgttcggcggcggcaccaagctgaccgtgctgggc

SEQ ID NO: 143 - DNA, 336 bp, Homo sapiens
  source 1..336: mol_type = unassigned DNA
cagagcgtgctgacccagccccccagcgtgagcggcgcccccggccagcgcgtgaccatcagctgcaccggcagcagcagcaacatcggcagcggctacgacgtgcactggtaccagcagctgcccggcaccgcccccaagctgctgatctacggcaacagcaagcgccccagcggcgtgcccgaccgcttcagcggcagcaagagcggcaccagcgccagcctggccatcaccggcctccagagcgaggacgaggccgactactactgtgccagctggaccgacggcctgagcctggtggtgttcggcggcggcaccaagctgaccgtgctgggc

SEQ ID NO: 144 - DNA, 336 bp, Homo sapiens
  source 1..336: mol_type = unassigned DNA
cagagcgtgctgacccagccgccttcagtgagtggcgcaccaggtcagcgtgtgaccatctcgtgtacgggcagcagcagcaacattggttctggttatgatgtgcattggtaccagcagttgcccgggacggcgccgaaacttctgatttatggtaattctaagcgtccctcaggcgtgccggatcgttttagcggatccaaaagcggcaccagcgcgagccttgcgattacgggcctgcaaagcgaagacgaagcggattattattgcgcttcttggactgatggtctttctcttgttgtgtttggcggcggcacgaagttaaccgttcttggc

SEQ ID NO: 145 - protein, 189 aa, Homo sapiens [sequence omitted]

SEQ ID NO: 146 - protein, 19 aa, Homo sapiens
NIEHKYLGYATSYAASVKG

SEQ ID NO: 147 - protein, 123 aa, Homo sapiens
QVQLVESGGGLVQPGGSLRLSCAASGFTFSSFGMSWVRQAPGKGLEWVSNIEHKYLGYATSYAASVKGRFTISRDNSKNTLYLQMNSLRAEDTAVYYCARYWGTPYLMQFDNWGQGTLVTVSS

SEQ ID NO: 148 - protein, 12 aa, Homo sapiens
HQGLIFYEKLLG

(19) United States
(12) Patent Application Publication
(10) Pub. No.: US 2025/0090632 A1
(43) Pub. Date: Mar. 20, 2025

(54) Methods of Treating Crohn's Disease with Anti-IL23 Specific Antibody

(71) Applicant: Janssen Biotech, Inc., Horsham, PA (US)

(72) Inventors: Omoniyi Adedokun, Phoenixville, PA (US); Daphane Chan, Abington, PA (US); Yang Chen, Ambler, PA (US); Philippe Szapary, Wynnewood, PA (US)

(21) Appl. No.: 18/908,031

(22) Filed: Oct. 7, 2024

Related U.S. Application Data
(63) Continuation of application No. 17/212,424, filed on Mar. 25, 2021, now Pat. No. 12,138,295.
Application No. 17/212,424 is a continuation of application No. 16/291,310, filed on Mar. 4, 2019, now abandoned.
(60) Provisional application No. 62/638,624, filed on Mar. 5, 2018.

(51) Int. Cl. A61K 38/17; A61K 39/00; A61P 1/00; A61P 37/06; C07K 16/24

(52) U.S. Cl. CPC A61K 38/1793; A61P 1/00; A61P 37/06; C07K 16/244; A61K 2039/505; A61K 2039/545; A61K 2039/57; C07K 2317/76

(57) ABSTRACT

A method of treating Crohn's disease in a patient administers an IL-23 specific antibody, e.g., guselkumab, at an initial intravenous dose and subsequence subcutaneous doses.

### CROSS-REFERENCE TO RELATED APPLICATIONS

This application is a continuation of U.S. patent application Ser. No. 17/212,424, filed 25 Mar. 2021, which is a continuation of U.S. patent application Ser. No. 16/291,310, filed 4 Mar. 2019, which claims priority to U.S. Provisional Application Ser. No. 62/638,624, filed 5 Mar. 2018. The entire contents of the aforementioned application are incorporated herein by reference in their entireties.

### SEQUENCE LISTING

The Sequence Listing written in the xml file titled: “206389-0037-02US_Replacement_Sequence_Listing.xml”; created on Nov. 6, 2024, and 150,445 bytes in size, is hereby incorporated by reference.

### FIELD OF THE INVENTION

The present invention concerns methods for treating Crohn's Disease with an antibody that binds the human IL-23 protein. In particular, it relates to dosing regimens for administration of an anti-IL-23 specific antibody and specific pharmaceutical compositions of an antibody, e.g., guselkumab.

### BACKGROUND OF THE INVENTION

Interleukin (IL)-12 is a secreted heterodimeric cytokine comprised of 2 disulfide-linked glycosylated protein subunits, designated p35 and p40 for their approximate molecular weights. IL-12 is produced primarily by antigen-presenting cells and drives cell-mediated immunity by binding to a two-chain receptor complex that is expressed on the surface of T cells or natural killer (NK) cells. The IL-12 receptor beta-1 (IL-12Rβ1) chain binds to the p40 subunit of IL-12, providing the primary interaction between IL-12 and its receptor. However, it is IL-12p35 ligation of the second receptor chain, IL-12Rβ2, that confers intracellular signaling (e.g. STAT4 phosphorylation) and activation of the receptor-bearing cell (Presky et al, 1996). IL-12 signaling concurrent with antigen presentation is thought to invoke T cell differentiation towards the T helper 1 (Th1) phenotype, characterized by interferon gamma (IFNγ) production (Trinchieri, 2003). Th1 cells are believed to promote immunity to some intracellular pathogens, generate complement-fixing antibody isotypes, and contribute to tumor immunosurveillance. Thus, IL-12 is thought to be a significant component to host defense immune mechanisms.

It was discovered that the p40 protein subunit of IL-12 can also associate with a separate protein subunit, designated p19, to form a novel cytokine, IL-23 (Oppman et al, 2000). IL-23 also signals through a two-chain receptor complex. Since the p40 subunit is shared between IL-12 and IL-23, it follows that the IL-12Rβ1 chain is also shared between IL-12 and IL-23. However, it is the IL-23p19 ligation of the second component of the IL-23 receptor complex, IL-23R, that confers IL-23 specific intracellular signaling (e.g., STAT3 phosphorylation) and subsequent IL-17 production by T cells (Parham et al, 2002; Aggarwal et al. 2003). Recent studies have demonstrated that the biological functions of IL-23 are distinct from those of IL-12, despite the structural similarity between the two cytokines (Langrish et al, 2005).

Abnormal regulation of IL-12 and Th1 cell populations has been associated with many immune-mediated diseases since neutralization of IL-12 by antibodies is effective in treating animal models of psoriasis, multiple sclerosis (MS), rheumatoid arthritis, inflammatory bowel disease, insulin-dependent (type 1) diabetes mellitus, and uveitis (Leonard et al, 1995; Hong et al, 1999; Malfait et al, 1998; Davidson et al, 1998). However, since these studies targeted the shared p40 subunit, both IL-12 and IL-23 were neutralized in vivo. Therefore, it was unclear whether IL-12 or IL-23 was mediating disease, or if both cytokines needed to be inhibited to achieve disease suppression. Recent studies have confirmed through IL-23p19 deficient mice or specific antibody neutralization of IL-23 that IL-23 inhibition can provide equivalent benefit as anti-IL-12p40 strategies (Cua et al, 2003, Murphy et al, 2003, Benson et al 2004). Therefore, there is increasing evidence for the specific role of IL-23 in immune-mediated disease. Neutralization of IL-23 without inhibition of IL-12 pathways could then provide effective therapy of immune-mediated disease with limited impact on important host defense immune mechanism. This would represent a significant improvement over current therapeutic options.

Currently, there are three classes of biologic agents approved for the treatment of moderately to severely active Crohn's disease: tumor necrosis factor (TNF) antagonist therapies (infliximab, adalimumab, certolizumab), integrin inhibitors (natalizumab and vedolizumab), and an IL-12/23 inhibitor (ustekinumab). Although the introduction of biologic agents has significantly improved the clinical management of patients with moderately to severely active Crohn's disease, a sizable proportion of the target patient population is non-responsive or will lose response over time. A review of the available data for approved biologic agents highlighted the unmet need in achieving and maintaining long-term remission, especially among patients who have previously failed biologic treatments. In all-treated patients (ie, all patients who were randomized at Week 0 of the studies evaluated), the estimated rates of clinical remission at 1 year in the biologic failure or intolerance (BIO-Failure) population is around 20%, and ranges from 20% to 50% in the conventional therapy failure or intolerance (CON-Failure) population.

In summary, there remains considerable unmet medical need for new treatment options, especially therapies with novel mechanisms of action that have the potential to raise the efficacy bar and maximize the proportion of patients who achieve and maintain clinical remission.

### SUMMARY OF THE INVENTION

In a first aspect, the invention concerns a method of a subject suffering from Crohn's disease comprising administering an anti-IL-23 specific antibody (also referred to as IL-23p19 antibody), e.g., guselkumab, to the patient in an initial intravenous induction dose from the start of treatment until 8 weeks from the start of treatment, and then subcutaneously administering the anti-IL-23 specific antibody once every 4 or 8 weeks thereafter, e.g., a dose at 0, 4, 8, 12 or 16, 20 or 24, 28 or 32, 36 or 40, 44 or 48 weeks. In addition, in another embodiment the subcutaneous treatment continues through 140 weeks after the start of treatment.

In one embodiment, the subject receives the anti-IL-23 specific antibody at a dose of 1200, 600 or 200 mg intravenously initially, 4 weeks after the initial dose and 8 weeks after the initial dose and continue with subcutaneous treatment of the anti-IL-23 specific antibody at 100 or 200 mg every 4 weeks through 44 weeks after initial treatment.

In another aspect, the composition used in the method of the invention comprises a pharmaceutical composition comprising: an anti-IL-23 specific antibody. In a preferred embodiment, the anti-IL-23 specific antibody is guselkumab in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

In an embodiment, Crohn's disease patients achieved significant improvement in clinical endpoints selected from:
(i) Change from Baseline in the Crohn® s Disease Activity Index (CDAI) Score at Week 12 The CDAI score will be assessed by collecting information on 8 different Crohn's disease-related variables, with scores ranging from 0 to approximately 600. A decrease over time indicates improvement in disease activity.
(ii) Clinical remission at Week 12, defined as CDAI less than (<) 150 points.
(iii) Clinical response at Week 12, defined as greater than or equal to (>=) 100-point reduction from baseline in CDAI score or (CDAI score<150.
(iv) Patient-Reported Outcome (PRO)-2 Remission at Week 12 defined based on average daily stool frequency (SF) and average daily abdominal pain (AP) score.
(v) Clinical-Biomarker Response at Week 12 defined using clinical response based on the CDAI score and reduction from baseline in C-reactive protein (CRP) or fecal calprotectin.
(vi) Endoscopic Response at Week 12 measured by the Simple Endoscopic Score for Crohn's Disease (SES-CD). The SES-CD is based on the evaluation of 4 endoscopic components across 5 ileocolonic segments, with a total score ranging from 0 to 56.
(vii) Clinical remission at Week 48 defined as CDAI score<150.
(viii) Durable Clinical Remission at Week 48 defined as CDAI<150 for most of all visits between Week 12 and Week 48.
(ix) Corticosteroid-Free Clinical Remission at Week 48 defined as CDAI score<150 at Week 48 and not receiving corticosteroids at Week 48.
(x) PRO-2 remission at Week 48 defined based on average daily stool frequency (SF) and average daily abdominal pain (AP) score. Fatigue response at Week 12 based on the Patient-Reported Outcomes Measurement Information System (PROMIS). Fatigue Short Form 7a contains 7 items that evaluate the severity of fatigue, with higher scores indicating greater fatigue.
(xi) Endoscopic response at Week 48 measured by the Simple Endoscopic Score for Crohn's Disease (SES-CD).

In another aspect of the invention the pharmaceutical composition comprises an isolated anti-IL23 specific antibody having the guselkumab CDR sequences comprising (i) the heavy chain CDR amino acid sequences of SEQ ID NO: 5, SEQ ID NO: 20, and SEQ ID NO: 44; and (ii) the light chain CDR amino acid sequences of SEQ ID NO: 50, SEQ ID NO: 56, and SEQ ID NO: 73 in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

Another aspect of the method of the invention comprises administering a pharmaceutical composition comprising an isolated anti-IL-23 specific antibody having the guselkumab heavy chain variable region amino acid sequence of SEQ ID NO: 106 and the guselkumab light chain variable region amino acid sequence of SEQ ID NO: 116 in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

### DETAILED DESCRIPTION OF THE PREFERRED EMBODIMENTS

As used herein the method of treatment of a subject suffering from Crohn's disease comprises administering isolated, recombinant and/or synthetic anti-IL-23 specific human antibodies and diagnostic and therapeutic compositions, methods and devices.

As used herein, an “anti-IL-23 specific antibody,” “anti-IL-23 antibody,” “antibody portion,” or “antibody fragment” and/or “antibody variant” and the like include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an IL-23 receptor or binding protein, which can be incorporated into an antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one IL-23 activity or binding, or with IL-23 receptor activity or binding, in vitro, in situ and/or in vivo. As a non-limiting example, a suitable anti-IL-23 antibody, specified portion or variant of the present invention can bind at least one IL-23 molecule, or specified portions, variants or domains thereof. A suitable anti-IL-23 antibody, specified portion, or variant can also optionally affect at least one of IL-23 activity or function, such as but not limited to, RNA, DNA or protein synthesis, IL-23 release, IL-23 receptor signaling, membrane IL-23 cleavage, IL-23 activity, IL-23 production and/or synthesis.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-23. For example, antibody fragments capable of binding to IL-23 or portions thereof, including, but not limited to, Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the C_{H}1 domain and/or hinge region of the heavy chain. The various portions of antibodies can be joined together chemically by conventional techniques, or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A “human antibody” may also be an antibody that is derived from or closely matches human germline immunoglobulin sequences. Human antibodies may include amino acid residues not encoded by germline immunoglobulin sequences (e.g., mutations introduced by random or site-specific mutagenesis in vitro or by somatic mutation in vivo). Often, this means that the human antibody is substantially non-immunogenic in humans. Human antibodies have been classified into groupings based on their amino acid sequence similarities. Accordingly, using a sequence similarity search, an antibody with a similar linear sequence can be chosen as a template to create a human antibody. Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. Thus, a human antibody is distinct from a chimeric or humanized antibody.

It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Bispecific, heterospecific, heteroconjugate or similar antibodies can also be used that are monoclonal, preferably, human or humanized, antibodies that have binding specificities for at least two different antigens. In the present case, one of the binding specificities is for at least one IL-23 protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), each entirely incorporated herein by reference.

Anti-IL-23 specific (also termed IL-23 specific antibodies) (or antibodies to IL-23) useful in the methods and compositions of the present invention can optionally be characterized by high affinity binding to IL-23 and, optionally and preferably, having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference). “Low immunogenicity” can also be defined as the incidence of titrable levels of antibodies to the anti-IL-23 antibody in patients treated with anti-IL-23 antibody as occurring in less than 25% of patients treated, preferably, in less than 10% of patients treated with the recommended dose for the recommended course of therapy during the treatment period.

The term “safe,” as it relates to a dose, dosage regimen, treatment or method with an anti-IL-23 antibody of the present invention (e.g., the anti-IL-23 antibody guselkumab), refers to a relatively low or reduced frequency and/or low or reduced severity of treatment-emergent adverse events (referred to as AEs or TEAEs) from the clinical trials conducted, e.g., Phase 2 clinical trials and earlier, compared to the standard of care or to another comparator. An adverse event is an untoward medical occurrence in a patient administered a medicinal product. In particular, safe as it relates to a dose, dosage regimen or treatment with an anti-IL-23 antibody of the present invention refers to a relatively low or reduced frequency and/or low or reduced severity of adverse events associated with administration of the antibody if attribution is considered to be possible, probable, or very likely due to the use of the anti-IL-23 antibody.

### Utility

The isolated nucleic acids of the present invention can be used for production of at least one anti-IL-23 antibody or specified variant thereof, which can be used to measure or effect in a cell, tissue, organ or animal (including mammals and humans), to diagnose, monitor, modulate, treat, alleviate, help prevent the incidence of, or reduce the symptoms of Crohn's disease.

Such a method can comprise administering an effective amount of a composition or a pharmaceutical composition comprising at least one anti-IL-23 antibody to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

### CITATIONS

All publications or patents cited herein, whether or not specifically designated, are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, NY (1989); Harlow and Lane, antibodies, a Laboratory Manual, Cold Spring Harbor, NY (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

### Antibodies of the Present Invention—Production and Generation

At least one anti-IL-23 antibody used in the method of the present invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, NY (1989); Harlow and Lane, antibodies, a Laboratory Manual, Cold Spring Harbor, NY (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001), each entirely incorporated herein by reference.

A preferred anti-IL-23 antibody is guselkumab (also referred to as CNTO1959) having the heavy chain variable region amino acid sequence of SEQ ID NO: 106 and the light chain variable region amino acid sequence of SEQ ID NO: 116 and having the heavy chain CDR amino acid sequences of SEQ ID NO: 5, SEQ ID NO: 20, and SEQ ID NO: 44; and the light chain CDR amino acid sequences of SEQ ID NO: 50, SEQ ID NO: 56, and SEQ ID NO: 73. Other anti-IL-23 antibodies have sequences listed herein and are described in U.S. Pat. No. 7,935,344, the entire contents of which are incorporated herein by reference).

Human antibodies that are specific for human IL-23 proteins or fragments thereof can be raised against an appropriate immunogenic antigen, such as an isolated IL-23 protein and/or a portion thereof (including synthetic molecules, such as synthetic peptides). Other specific or general mammalian antibodies can be similarly raised. Preparation of immunogenic antigens, and monoclonal antibody production can be performed using any suitable technique.

In one approach, a hybridoma is produced by fusing a suitable immortal cell line (e.g., a myeloma cell line, such as, but not limited to, Sp2/0, Sp2/0-AG14, NSO, NS1, NS2, AE-1, L.5, L243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMALWA, NEURO 2A, or the like, or heteromylomas, fusion products thereof, or any cell or fusion cell derived therefrom, or any other suitable cell line as known in the art) (see, e.g., www.atcc.org, www.lifetech.com., and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably, the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, CA; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260(5/12/94); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, predecessor of Applied Molecular Evolution (AME), each entirely incorporated herein by reference)) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, MA; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammal. These non-human amino acid residues are replaced by residues often referred to as “import” residues, which are typically taken from an “import” variable, constant or other domain of a known human sequence.

Known human Ig sequences are disclosed, e.g., www.ncbi.nlm.nih.gov/entrez/query.fcgi; www.ncbi.nih.gov/igblast; www.atcc.org/phage/hdb.html; www.mrc-cpe.cam.ac.uk/ALIGNMENTS.php; www.kabatdatabase.com/top.html; ftp.ncbi.nih.gov/repository/kabat; www.sciquest.com; www.abcam.com; www.antibodyresource.com/onlinecomp.html; www.public.iastate.edu/˜pedro/research_tools.html; www.whfreeman.com/immunology/CH05/kuby05.htm; www.hhmi.org/grants/lectures/1996/vlab; www.path.cam.ac.uk/˜mrc7/mikeimages.html; mcb.harvard.edu/BioLinks/Immunology.html; www.immunologylink.com; pathbox.wustl.edu/˜hcenter/index.html; www.appliedbiosystems.com; www.nal.usda.gov/awic/pubs/antibody; www.m.ehime-u.ac.jp/˜yasuhito/Elisa.html; www.biodesign.com; www.cancerresearchuk.org; www.biotech.ufl.edu; www.isac-net.org; baserv.uci.kun.nl/˜jraats/linksl.html; www.recab.uni-hd.de/immuno.bme.nwu.edu; www.mrc-cpe.cam.ac.uk; www.ibt.unam.mx/vir/V_mice.html; http.//www.bioinforg.uk/abs; antibody.bath.ac.uk; www.unizh.ch; www.cryst.bbk.ac.uk/˜ubcg07s; www.nimr.mrc.ac.uk/CC/ccaewg/ccaewg.html; www.path.cam.ac.uk/˜mrc7/humanisation/TATHIP.html; www.ibt.unam.mx/vir/structure/stat_aim.html; www.biosci.missouri.edu/smithgp/index.html; www.jerini.de; Kabat et al., Sequences of Proteins of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing antigen binding. Accordingly, part or all of the non-human or human CDR sequences are maintained while the non-human sequences of the variable and constant regions may be replaced with human or other amino acids.

Antibodies can also optionally be humanized or human antibodies engineered with retention of high affinity for the antigen and other favorable biological properties. To achieve this goal, humanized (or human) antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In addition, the human IL-23 specific antibody used in the method of the present invention may comprise a human germline light chain framework. In particular embodiments, the light chain germline sequence is selected from human VK sequences including, but not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8. In certain embodiments, this light chain human germline framework is selected from V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6.

In other embodiments, the human IL-23 specific antibody used in the method of the present invention may comprise a human germline heavy chain framework. In particular embodiments, this heavy chain human germline framework is selected from VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, V14-28, VH4-31, VH4-34, VH4-39, V14-4, VH4-59, VH4-61, VH5-51, VH6-1, and VH7-81.

In particular embodiments, the light chain variable region and/or heavy chain variable region comprises a framework region or at least a portion of a framework region (e.g., containing 2 or 3 subregions, such as FR2 and FR3). In certain embodiments, at least FRL1, FRL2, FRL3, or FRL4 is fully human. In other embodiments, at least FRH1, FRH2, FRH3, or FRH4 is fully human. In some embodiments, at least FRL1, FRL2, FRL3, or FRL4 is a germline sequence (e.g., human germline) or comprises human consensus sequences for the particular framework (readily available at the sources of known human Ig sequences described above). In other embodiments, at least FRH1, FRH2, FRH3, or FRH4 is a germline sequence (e.g., human germline) or comprises human consensus sequences for the particular framework. In preferred embodiments, the framework region is a fully human framework region.

Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766886, 5714352, 6204023, 6180370, 5693762, 5530101, 5585089, 5225539; 4816567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.

In certain embodiments, the antibody comprises an altered (e.g., mutated) Fc region. For example, in some embodiments, the Fc region has been altered to reduce or enhance the effector functions of the antibody. In some embodiments, the Fc region is an isotype selected from IgM, IgA, IgG, IgE, or other isotype. Alternatively or additionally, it may be useful to combine amino acid modifications with one or more further amino acid modifications that alter C1q binding and/or the complement dependent cytotoxicity function of the Fc region of an IL-23 binding molecule. The starting polypeptide of particular interest may be one that binds to C1q and displays complement dependent cytotoxicity (CDC). Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO0042072, which is hereby incorporated by reference.

As disclosed above, one can design an Fc region of the human IL-23 specific antibody of the present invention with altered effector function, e.g., by modifying C1q binding and/or FcγR binding and thereby changing complement dependent cytotoxicity (CDC) activity and/or antibody-dependent cell-mediated cytotoxicity (ADCC) activity. “Effector functions” are responsible for activating or diminishing a biological activity (e.g., in a subject). Examples of effector functions include, but are not limited to: C1q binding; CDC; Fc receptor binding; ADCC; phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of the human IL-23 (or anti-IL-23) antibody with improved C1q binding and improved FcγRIIIbinding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

Fc mutations can also be introduced in engineer to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, J. Biol. Chem. 276:6591-6604).

Another type of amino acid substitution serves to alter the glycosylation pattern of the Fc region of the human IL-23 specific antibody. Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation sites that are not present in the polypeptide. Addition of glycosylation sites to the Fc region of a human IL-23 specific antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the human IL-23 specific antibody of the present invention is expressed in cells that express beta (1,4)—N-acetylglucosaminyltransferase III (GnT III), such that GnT III adds GlcNAc to the human IL-23 antibody. Methods for producing antibodies in such a fashion are provided in WO/9954342, WO/03011878, patent publication 20030003097A1, and Umana et al., Nature Biotechnology, 17:176-180, February 1999; all of which are herein specifically incorporated by reference in their entireties.

The anti-IL-23 antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-IL-23 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, CA), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, 5427908, 5580717, assigned to Affymax; 5885793, assigned to Cambridge antibody Technologies; 5750373, assigned to Genentech, 5618920, 5595898, 5576195, 5698435, 5693493, 5698417, assigned to Xoma, Colligan, supra; Ausubel, supra; or Sambrook, supra, each of the above patents and publications entirely incorporated herein by reference.

Antibodies used in the method of the present invention can also be prepared using at least one anti-IL23 antibody encoding nucleic acid to provide transgenic animals or mammals, such as goats, cows, horses, sheep, rabbits, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies used in the method of the present invention can additionally be prepared using at least one anti-IL23 antibody encoding nucleic acid to provide transgenic plants and cultured plant cells (e.g., but not limited to, tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and references cited therein. Thus, antibodies of the present invention can also be produced using transgenic plants, according to known methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

The antibodies used in the method of the invention can bind human IL-23 with a wide range of affinities (K_{D}). In a preferred embodiment, a human mAb can optionally bind human IL-23 with high affinity. For example, a human mAb can bind human IL-23 with a K_{D} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, NY (1984); Kuby, Janis Immunology, W. H. Freeman and Company: New York, NY (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

### Nucleic Acid Molecules

Using the information provided herein, for example, the nucleotide sequences encoding at least 70-100% of the contiguous amino acids of at least one of the light or heavy chain variable or CDR regions described herein, among other sequences disclosed herein, specified fragments, variants or consensus sequences thereof, or a deposited vector comprising at least one of these sequences, a nucleic acid molecule of the present invention encoding at least one anti-IL-23 antibody can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules used in the method of the present invention can include nucleic acid molecules comprising an open reading frame (ORF), optionally, with one or more introns, e.g., but not limited to, at least one specified portion of at least one CDR, such as CDR1, CDR2 and/or CDR3 of at least one heavy chain or light chain; nucleic acid molecules comprising the coding sequence for an anti-IL-23 antibody or variable region; and nucleic acid molecules which comprise a nucleotide sequence substantially different from those described above but which, due to the degeneracy of the genetic code, still encode at least one anti-IL-23 antibody as described herein and/or as known in the art. Of course, the genetic code is well known in the art. Thus, it would be routine for one skilled in the art to generate such degenerate nucleic acid variants that code for specific anti-IL-23 antibodies used in the method of the present invention. See, e.g., Ausubel, et al., supra, and such nucleic acid variants are included in the present invention. Non-limiting examples of isolated nucleic acid molecules include nucleic acids encoding HC CDR1, HC CDR2, HC CDR3, LC CDR1, LC CDR2, and LC CDR3, respectively.

As indicated herein, nucleic acid molecules which comprise a nucleic acid encoding an anti-IL-23 antibody can include, but are not limited to, those encoding the amino acid sequence of an antibody fragment, by itself, the coding sequence for the entire antibody or a portion thereof; the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

### Polynucleotides Selectively Hybridizing to a Polynucleotide as Described Herein

The method of the present invention uses isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of an antibody. The polynucleotides embrace nucleic acid sequences that can be employed for selective hybridization to a polynucleotide encoding an antibody of the present invention. See, e.g., Ausubel, supra; Colligan, supra, each entirely incorporated herein by reference.

### Construction of Nucleic Acids

The isolated nucleic acids can be made using (a) recombinant methods, (b) synthetic techniques, (c) purification techniques, and/or (d) combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention, excluding the coding sequence, is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

### Recombinant Methods for Constructing Nucleic Acids

The isolated nucleic acid compositions, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, are well known to those of ordinary skill in the art. (See, e.g., Ausubel, supra; or Sambrook, supra)

### Nucleic Acid Screening and Isolation Methods

A cDNA or genomic library can be screened using a probe based upon the sequence of a polynucleotide used in the method of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides used in the method of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, CA (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids used in the method of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The present invention uses recombinant expression cassettes comprising a nucleic acid. A nucleic acid sequence, for example, a cDNA or a genomic sequence encoding an antibody used in the method of the present invention, can be used to construct a recombinant expression cassette that can be introduced into at least one desired host cell. A recombinant expression cassette will typically comprise a polynucleotide operably linked to transcriptional initiation regulatory sequences that will direct the transcription of the polynucleotide in the intended host cell. Both heterologous and non-heterologous (i.e., endogenous) promoters can be employed to direct expression of the nucleic acids.

In some embodiments, isolated nucleic acids that serve as promoter, enhancer, or other elements can be introduced in the appropriate position (upstream, downstream or in the intron) of a non-heterologous form of a polynucleotide of the present invention so as to up or down regulate expression of a polynucleotide. For example, endogenous promoters can be altered in vivo or in vitro by mutation, deletion and/or substitution.

### Vectors and Host Cells

The present invention also relates to vectors that include isolated nucleic acid molecules, host cells that are genetically engineered with the recombinant vectors, and the production of at least one anti-IL-23 antibody by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one antibody used in the method of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein used in the method of the present invention. Alternatively, nucleic acids can be expressed in a host cell by turning on (by manipulation) in a host cell that contains endogenous DNA encoding an antibody. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Purification of an Antibody

An anti-IL-23 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, NY, (1997-2001), e.g., Chapters 1, 4, 6, 8, 9, 10, each entirely incorporated herein by reference.

Antibodies used in the method of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Anti-IL-23 Antibodies.

An anti-IL-23 antibody according to the present invention includes any protein or peptide containing molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, further optionally comprising at least one substitution, insertion or deletion), a heavy chain or light chain constant region, (e.g., comprising at least one C_{H}1, hinge1, hinge2, hinge3, hinge4, C_{H}2, or C_{H}3 or fragment thereof, further optionally comprising at least one substitution, insertion or deletion), or any portion thereof, that can be incorporated into an antibody. An antibody can include or be derived from any mammal, such as but not limited to, a human, a mouse, a rabbit, a rat, a rodent, a primate, or any combination thereof, and the like.

The isolated antibodies used in the method of the present invention comprise the antibody amino acid sequences disclosed herein encoded by any suitable polynucleotide, or any isolated or prepared antibody. Preferably, the human antibody or antigen-binding fragment binds human IL-23 and, thereby, partially or substantially neutralizes at least one biological activity of the protein. An antibody, or specified portion or variant thereof, that partially or preferably substantially neutralizes at least one biological activity of at least one IL-23 protein or fragment can bind the protein or fragment and thereby inhibit activities mediated through the binding of IL-23 to the IL-23 receptor or through other IL-23-dependent or mediated mechanisms. As used herein, the term “neutralizing antibody” refers to an antibody that can inhibit an IL-23-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-23 antibody to inhibit an IL-23-dependent activity is preferably assessed by at least one suitable IL-23 protein or receptor assay, as described herein and/or as known in the art. A human antibody can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, γ4). Antibodies of this type can be prepared by employing a transgenic mouse or other transgenic non-human mammal comprising at least one human light chain (e.g., IgG, IgA, and IgM) transgenes as described herein and/or as known in the art. In another embodiment, the anti-IL-23 human antibody comprises an IgG1 heavy chain and an IgG1 light chain.

An antibody binds at least one specified epitope specific to at least one IL-23 protein, subunit, fragment, portion or any combination thereof. The at least one epitope can comprise at least one antibody binding region that comprises at least one portion of the protein, which epitope is preferably comprised of at least one extracellular, soluble, hydrophillic, external or cytoplasmic portion of the protein.

Generally, the human antibody or antigen-binding fragment will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences may be derived from human germline sequences or closely match the germline sequences. For example, the CDRs from a synthetic library derived from the original non-human CDRs can be used. These CDRs may be formed by incorporation of conservative substitutions from the original non-human sequence. In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3.

Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

The anti-IL-23 specific antibody can comprise at least one of a heavy or light chain variable region having a defined amino acid sequence. For example, in a preferred embodiment, the anti-IL-23 antibody comprises at least one of at least one heavy chain variable region, optionally having the amino acid sequence of SEQ ID NO:106 and/or at least one light chain variable region, optionally having the amino acid sequence of SEQ ID NO:116. Antibodies that bind to human IL-23 and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human IL-23 or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

The invention also relates to antibodies, antigen-binding fragments, immunoglobulin chains and CDRs comprising amino acids in a sequence that is substantially the same as an amino acid sequence described herein. Preferably, such antibodies or antigen-binding fragments and antibodies comprising such chains or CDRs can bind human IL-23 with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include, without limitation, replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

### Amino Acid Codes

The amino acids that make up anti-IL-23 antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994):

| SINGLE | THREE | | |
|---|---|---|---|
| LETTER | LETTER | | THREE NUCLEOTIDE |
| CODE | CODE | NAME | CODON(S) |
| A | Ala | Alanine | GCA, GCC, GCG, GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, CUC, |
| | | | CUG, CUU |
| M | Met | Methionine | AUG |
| N | Asn | Asparagine | AAC, AAU |
| P | Pro | Proline | CCA, CCC, CCG, CCU |
| Q | Gln | Glutamine | CAA, CAG |
| R | Arg | Arginine | AGA, AGG, CGA, CGC, |
| | | | CGG, CGU |
| S | Ser | Serine | AGC, AGU, UCA, UCC, |
| | | | UCG, UCU |
| T | Thr | Threonine | ACA, ACC, ACG, ACU |
| V | Val | Valine | GUA, GUC, GUG, GUU |
| W | Trp | Tryptophan | UGG |
| Y | Tyr | Tyrosine | UAC, UAU |

An anti-IL-23 antibody used in the method of the present invention can include one or more amino acid substitutions, deletions or additions, either from natural mutations or human manipulation, as specified herein.

The number of amino acid substitutions a skilled artisan would make depends on many factors, including those described above. Generally speaking, the number of amino acid substitutions, insertions or deletions for any given anti-IL-23 antibody, fragment or variant will note more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an anti-IL-23 specific antibody that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one IL-23 neutralizing activity. Sites that are critical for antibody binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

Anti-IL-23 antibodies can include, but are not limited to, at least one portion, sequence or combination selected from 5 to all of the contiguous amino acids of at least one of SEQ ID NOS: 5, 20, 44, 50, 56, and 73.

IL-23 antibodies or specified portions or variants can include, but are not limited to, at least one portion, sequence or combination selected from at least 3-5 contiguous amino acids of the SEQ ID NOs above; 5-17 contiguous amino acids of the SEQ ID NOs above, 5-10 contiguous amino acids of the SEQ ID NOs above, 5-11 contiguous amino acids of the SEQ ID NOs above, 5-7 contiguous amino acids of the SEQ ID NOs above; 5-9 contiguous amino acids of the SEQ ID NOs above.

An anti-IL-23 antibody can further optionally comprise a polypeptide of at least one of 70-100% of 5, 17, 10, 11, 7, 9, 119, or 108 contiguous amino acids of the SEQ ID NOs above. In one embodiment, the amino acid sequence of an immunoglobulin chain, or portion thereof (e.g., variable region, CDR) has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of the SEQ ID NOs above. For example, the amino acid sequence of a light chain variable region can be compared with the sequence of the SEQ ID NOs above, or the amino acid sequence of a heavy chain CDR3 can be compared with the SEQ ID NOs above. Preferably, 70-100% amino acid identity (i.e., 90, 91, 92, 93, 94, 95, 96, 97, 98, 99, 100 or any range or value therein) is determined using a suitable computer algorithm, as known in the art.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, MD).

Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

Preferred parameters for polypeptide sequence comparison include the following:
(1) Algorithm: Needleman and Wunsch, J. Mol Biol. 48:443-453 (1970) Comparison matrix: BLOSSUM62 from Hentikoff and Hentikoff, Proc. Natl. Acad. Sci, USA. 89:10915-10919 (1992)
Gap Penalty: 12
Gap Length Penalty: 4
A program useful with these parameters is publicly available as the “gap” program from Genetics Computer Group, Madison Wis. The aforementioned parameters are the default parameters for peptide sequence comparisons (along with no penalty for end gaps).

Preferred parameters for polynucleotide comparison include the following:
(1) Algorithm: Needleman and Wunsch, J. Mol Biol. 48:443-453 (1970)
Comparison matrix: matches=+10, mismatch=0
Gap Penalty: 50
Gap Length Penalty: 3
Available as: The “gap” program from Genetics Computer Group, Madison Wis. These are the default parameters for nucleic acid sequence comparisons.

By way of example, a polynucleotide sequence may be identical to another sequence, that is 100% identical, or it may include up to a certain integer number of nucleotide alterations as compared to the reference sequence. Such alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the sequence by the numerical percent of the respective percent identity (divided by 100) and subtracting that product from the total number of nucleotides in the sequence, or:
n.sub.n.ltorsim.x.sub.n−(x.sub.n.y),
   wherein n.sub.n is the number of nucleotide alterations, x.sub.n is the total number of nucleotides in sequence, and y is, for instance, 0.70 for 70%, 0.80 for 80%, 0.85 for 85%, 0.90 for 90%, 0.95 for 95%, etc., and wherein any non-integer product of x.sub.n and y is rounded down to the nearest integer prior to subtracting from x.sub.n.

Alterations of a polynucleotide sequence encoding the the SEQ ID NOs above may create nonsense, missense or frameshift mutations in this coding sequence and thereby alter the polypeptide encoded by the polynucleotide following such alterations. Similarly, a polypeptide sequence may be identical to the reference sequence of the SEQ ID NOs above, that is be 100% identical, or it may include up to a certain integer number of amino acid alterations as compared to the reference sequence such that the percentage identity is less than 100%. Such alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the SEQ ID NOs above by the numerical percent of the respective percent identity (divided by 100) and then subtracting that product from the total number of amino acids in the SEQ ID NOs above, or:
n.sub.a.ltorsim.x.sub.a−(x.sub.a.y),
   wherein n.sub.a is the number of amino acid alterations, x.sub.a is the total number of amino acids in the SEQ ID NOs above, and y is, for instance 0.70 for 70%, 0.80 for 80%, 0.85 for 85% etc., and wherein any non-integer produce of x.sub.a and y is rounded down to the nearest integer prior to subtracting it from x.sub.a.

Exemplary heavy chain and light chain variable regions sequences and portions thereof are provided in the SEQ ID NOs above. The antibodies of the present invention, or specified variants thereof, can comprise any number of contiguous amino acid residues from an antibody of the present invention, wherein that number is selected from the group of integers consisting of from 10-100% of the number of contiguous residues in an anti-IL-23 antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active antibody of the present invention. Biologically active antibodies have a specific activity at least 20%, 30%, or 40%, and, preferably, at least 50%, 60%, or 70%, and, most preferably, at least 80%, 90%, or 95%-100% or more (including, without limitation, up to 10 times the specific activity) of that of the native (non-synthetic), endogenous or related and known antibody. Methods of assaying and quantifying measures of enzymatic activity and substrate specificity are well known to those of skill in the art.

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C₁₂, laurate), n-tetradecanoate (C₁₄, myristate), n-octadecanoate (Cis, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, CA (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—.

Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified antibodies can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, CA (1996).

The method of the present invention also uses an anti-IL-23 antibody composition comprising at least one, at least two, at least three, at least four, at least five, at least six or more anti-IL-23 antibodies thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the anti-IL-23 antibody amino acid sequence selected from the group consisting of 70-100% of the contiguous amino acids of the SEQ ID NOs above, or specified fragments, domains or variants thereof. Preferred anti-IL-23 antibody compositions include at least one or two full length, fragments, domains or variants as at least one CDR or LBP containing portions of the anti-IL-23 antibody sequence described herein, for example, 70-100% of the SEQ ID NOs above, or specified fragments, domains or variants thereof. Further preferred compositions comprise, for example, 40-99% of at least one of 70-100% of the SEQ ID NOs above, etc., or specified fragments, domains or variants thereof. Such composition percentages are by weight, volume, concentration, molarity, or molality as liquid or dry solutions, mixtures, suspension, emulsions, particles, powder, or colloids, as known in the art or as described herein.

### Antibody Compositions Comprising Further Therapeutically Active Ingredients

The antibody compositions used in the method of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21^{st} edition, Springhouse Corp., Springhouse, PA, 2001; Health Professional's Drug Guide 2001, ed., Shannon, Wilson, Stang, Prentice-Hall, Inc, Upper Saddle River, NJ; Pharmcotherapy Handbook, Wells et al., ed., Appleton & Lange, Stamford, CT, each entirely incorporated herein by reference).

By way of example of the drugs that can be combined with the antibodies for the method of the present invention, the anti-infective drug can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives. The hormonal drug can be at least one selected from corticosteroids, androgens or at least one anabolic steroid, estrogen or at least one progestin, gonadotropin, antidiabetic drug or at least one glucagon, thyroid hormone, thyroid hormone antagonist, pituitary hormone, and parathyroid-like drug. The at least one cephalosporin can be at least one selected from cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef.

The at least one coricosteroid can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate. The at least one androgen or anabolic steroid can be at least one selected from danazol, fluoxymesterone, methyltestosterone, nandrolone decanoate, nandrolone phenpropionate, testosterone, testosterone cypionate, testosterone enanthate, testosterone propionate, and testosterone transdermal system.

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, and tacrolimus.

The at least one local anti-infective can be at least one selected from acyclovir, amphotericin B, azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. The at least one scabicide or pediculicide can be at least one selected from crotamiton, lindane, permethrin, and pyrethrins. The at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide. (See, e.g., pp. 1098-1136 of Nursing 2001 Drug Handbook.)

Anti-IL-23 antibody compositions can further comprise at least one of any suitable and effective amount of a composition or pharmaceutical composition comprising at least one anti-IL-23 antibody contacted or administered to a cell, tissue, organ, animal or patient in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), an immunization, an immunoglobulin, an immunosuppressive (e.g., basiliximab, cyclosporine, daclizumab), a cytokine or a cytokine antagonist. Non-limiting examples of such cytokines include, but are not limited to, any of IL-1 to IL-23 et al. (e.g., IL-1, IL-2, etc.). Suitable dosages are well known in the art. See, e.g., Wells et al., eds., Pharmacotherapy Handbook, 2^{nd} Edition, Appleton and Lange, Stamford, CT (2000); PDR Pharmacopoeia, Tarascon Pocket Pharmacopoeia 2000, Deluxe Edition, Tarascon Publishing, Loma Linda, CA (2000), each of which references are entirely incorporated herein by reference.

Anti-IL-23 antibody compounds, compositions or combinations used in the method of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, PA) 1990. Pharmaceutically acceptable carriers can be routinely selected that are suitable for the mode of administration, solubility and/or stability of the anti-IL-23 antibody, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL-23 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL-23 antibody compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL-23 antibody, portion or variant compositions according to the invention are known in the art, e.g., as listed in “Remington: The Science & Practice of Pharmacy,” 19^{th} ed., Williams & Williams, (1995), and in the “Physician's Desk Reference,” 52^{nd} ed., Medical Economics, Montvale, NJ (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

### Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL-23 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the method of the invention uses an article of manufacture, comprising packaging material and at least one vial comprising a solution of at least one anti-IL-23 specific antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further uses an article of manufacture, comprising packaging material, a first vial comprising lyophilized anti-IL-23 specific antibody, and a second vial comprising an aqueous diluent of prescribed buffer or preservative, wherein said packaging material comprises a label that instructs a patient to reconstitute the anti-IL-23 specific antibody in the aqueous diluent to form a solution that can be held over a period of twenty-four hours or greater.

The anti-IL-23 specific antibody used in accordance with the present invention can be produced by recombinant means, including from mammalian cell or transgenic preparations, or can be purified from other biological sources, as described herein or as known in the art.

The range of the anti-IL-23 specific antibody includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations can be prepared by a process which comprises mixing at least one anti-IL-23 specific antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL-23 specific antibody and preservative in an aqueous diluent is carried out using conventional dissolution and mixing procedures. To prepare a suitable formulation, for example, a measured amount of at least one anti-IL-23 specific antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The formulations can be provided to patients as clear solutions or as dual vials comprising a vial of lyophilized anti-IL-23 specific antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present articles of manufacture are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biologically activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of anti-IL-23 specific antibody can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one anti-IL-23 specific antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one anti-IL-23 specific antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising single vial systems include pen-injector devices for delivery of a solution, such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, Smartject® e.g., as made or developed by Becton Dickensen (Franklin Lakes, NJ, www.bectondickenson.com), Disetronic (Burgdorf, Switzerland, www.disetronic.com; Bioject, Portland, Oregon (www.bioject.com); National Medical Products, Weston Medical (Peterborough, UK, www.weston-medical.com), Medi-Ject Corp (Minneapolis, MN, www.mediject.com), and similarly suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors, and needle free IV infusion sets.

The products may include packaging material. The packaging material provides, in addition to the information required by the regulatory agencies, the conditions under which the product can be used. The packaging material of the present invention provides instructions to the patient, as applicable, to reconstitute the at least one anti-IL-23 antibody in the aqueous diluent to form a solution and to use the solution over a period of 2-24 hours or greater for the two vial, wet/dry, product. For the single vial, solution product, pre-filled syringe or auto-injector, the label indicates that such solution can be used over a period of 2-24 hours or greater. The products are useful for human pharmaceutical product use.

The formulations used in the method of the present invention can be prepared by a process that comprises mixing an anti-IL-23 antibody and a selected buffer, preferably, a phosphate buffer containing saline or a chosen salt. Mixing the anti-IL-23 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The method of the invention provides pharmaceutical compositions comprising various formulations useful and acceptable for administration to a human or animal patient. Such pharmaceutical compositions are prepared using water at “standard state” as the diluent and routine methods well known to those of ordinary skill in the art. For example, buffering components such as histidine and histidine monohydrochloride hydrate, may be provided first followed by the addition of an appropriate, non-final volume of water diluent, sucrose and polysorbate 80 at “standard state.” Isolated antibody may then be added. Last, the volume of the pharmaceutical composition is adjusted to the desired final volume under “standard state” conditions using water as the diluent. Those skilled in the art will recognize a number of other methods suitable for the preparation of the pharmaceutical compositions.

The pharmaceutical compositions may be aqueous solutions or suspensions comprising the indicated mass of each constituent per unit of water volume or having an indicated pH at “standard state.” As used herein, the term “standard state” means a temperature of 25° C.+/−2° C. and a pressure of 1 atmosphere. The term “standard state” is not used in the art to refer to a single art recognized set of temperatures or pressure, but is instead a reference state that specifies temperatures and pressure to be used to describe a solution or suspension with a particular composition under the reference “standard state” conditions. This is because the volume of a solution is, in part, a function of temperature and pressure. Those skilled in the art will recognize that pharmaceutical compositions equivalent to those disclosed here can be produced at other temperatures and pressures. Whether such pharmaceutical compositions are equivalent to those disclosed here should be determined under the “standard state” conditions defined above (e.g. 25° C.+/−2° C. and a pressure of 1 atmosphere).

Importantly, such pharmaceutical compositions may contain component masses “about” a certain value (e.g. “about 0.53 mg L-histidine”) per unit volume of the pharmaceutical composition or have pH values about a certain value. A component mass present in a pharmaceutical composition or pH value is “about” a given numerical value if the isolated antibody present in the pharmaceutical composition is able to bind a peptide chain while the isolated antibody is present in the pharmaceutical composition or after the isolated antibody has been removed from the pharmaceutical composition (e.g., by dilution). Stated differently, a value, such as a component mass value or pH value, is “about” a given numerical value when the binding activity of the isolated antibody is maintained and detectable after placing the isolated antibody in the pharmaceutical composition.

Competition binding analysis is performed to determine if the IL-23 specific mAbs bind to similar or different epitopes and/or compete with each other. Abs are individually coated on ELISA plates. Competing mAbs are added, followed by the addition of biotinylated hrIL-23. For positive control, the same mAb for coating may be used as the competing mAb (“self-competition”). IL-23 binding is detected using streptavidin. These results demonstrate whether the mAbs recognize similar or partially overlapping epitopes on IL-23.

One aspect of the method of the invention administers to a patient a pharmaceutical composition comprising

In one embodiment of the pharmaceutical compositions, the isolated antibody concentration is from about 77 to about 104 mg per ml of the pharmaceutical composition. In another embodiment of the pharmaceutical compositions the pH is from about 5.5 to about 6.5.

The stable or preserved formulations can be provided to patients as clear solutions or as dual vials comprising a vial of lyophilized at least one anti-IL-23 antibody that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the anti-IL-23 antibody may result in other than a clear solution of lyophilized powder comprising the antibody. Among non-clear solutions are formulations comprising particulate suspensions, said particulates being a composition containing the anti-IL-23 antibody in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gleatin agar, starch, arabinogalactan, albumin, collagen, polyglycolic acid, polylactic aced, glycolide-L(−) lactide poly(episilon-caprolactone, poly(epsilon-caprolactone-CO-lactic acid), poly(epsilon-caprolactone-CO-glycolic acid), poly(B-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(episilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried antibody preparation is taught in U.S. Pat. No. 6,019,968. The antibody-based dry powder compositions may be produced by spray drying solutions or slurries of the antibody and, optionally, excipients, in a solvent under conditions to provide a respirable dry powder. Solvents may include polar compounds, such as water and ethanol, which may be readily dried. Antibody stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

An anti-IL-23 antibody in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Therapeutic Applications

The present invention also provides a method for modulating or treating Crohn's disease, in a cell, tissue, organ, animal, or patient, as known in the art or as described herein, using at least one IL-23 antibody of the present invention, e.g., administering or contacting the cell, tissue, organ, animal, or patient with a therapeutic effective amount of IL-23 specific antibody.

Any method of the present invention can comprise administering an effective amount of a composition or pharmaceutical composition comprising an anti-IL-23 antibody to a cell, tissue, organ, animal or patient in need of such modulation, treatment or therapy. Such a method can optionally further comprise co-administration or combination therapy for treating such diseases or disorders, wherein the administering of said at least one anti-IL-23 antibody, specified portion or variant thereof, further comprises administering, before concurrently, and/or after, at least one selected from at least one TNF antagonist (e.g., but not limited to, a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept (Enbrel™), adalimulab (Humira™), CDP-571, CDP-870, afelimomab, lenercept, and the like), an antirheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, CT (2000); PDR Pharmacopoeia, Tarascon Pocket Pharmacopoeia 2000, Deluxe Edition, Tarascon Publishing, Loma Linda, CA (2000); Nursing 2001 Handbook of Drugs, 21st edition, Springhouse Corp., Springhouse, PA, 2001; Health Professional's Drug Guide 2001, ed., Shannon, Wilson, Stang, Prentice-Hall, Inc, Upper Saddle River, NJ, each of which references are entirely incorporated herein by reference.

### Therapeutic Treatments

Typically, treatment of Crohn's disease is affected by administering an effective amount or dosage of an anti-IL-23 antibody composition that total, on average, a range from at least about 0.01 to 500 milligrams of an anti-IL-23 antibody per kilogram of patient per dose, and, preferably, from at least about 0.1 to 100 milligrams antibody/kilogram of patient per single or multiple administration, depending upon the specific activity of the active agent contained in the composition. Alternatively, the effective serum concentration can comprise 0.1-5000 μg/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5,6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5., 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and, preferably, 0.1 to 10 milligrams per kilogram per administration or in sustained release form is effective to obtain desired results.

As a non-limiting example, treatment of humans or animals can be provided as a one-time or periodic dosage of at least one antibody of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or, alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or, alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the antibody can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Alternative Administration

Many known and developed modes can be used according to the present invention for administering pharmaceutically effective amounts of an anti-IL-23 antibody. While pulmonary administration is used in the following description, other modes of administration can be used according to the present invention with suitable results. IL-23 specific antibodies of the present invention can be delivered in a carrier, as a solution, emulsion, colloid, or suspension, or as a dry powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### Alternative Delivery

The invention further relates to the administration of an anti-IL-23 antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An anti-IL-23 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

Having generally described the invention, the same will be more readily understood by reference to the following Examples, which are provided by way of illustration and are not intended as limiting. Further details of the invention are illustrated by the following non-limiting Examples. The disclosures of all citations in the specification are expressly incorporated herein by reference.

### Example 1

Preclinical Evidence Implicating IL-23 as a Target in Crohn's Disease Genetic and animal model studies have explored the contribution of IL-12 and IL-23 in driving the pathophysiology of Crohn's disease. The results indicate that IL-23 plays a predominant role in inflammatory bowel disease (IBD) and emerging evidence suggests that blocking IL-23 alone may be a more effective strategy than blocking both IL-12 and IL-23.

Initial observations from genetic and animal model data suggest that Crohn's disease is mediated by IL-12 and/or IL-23, potentially through the Th1 and Th17 pathways they induce, respectively. However, increasing evidence suggests a predominant role for IL-23 in Crohn's disease. Genome-wide association studies identified polymorphisms in the IL-23R gene that are associated with Crohn's disease. The role of IL-23 in driving intestinal inflammation has been shown in several mouse models. Mice treated with anti-IL-23 antibodies exhibited attenuated inflammation, and mice with a genetic deletion of the p19 subunit of IL-23 are protected in several models of intestinal inflammation.

### Clinical Evidence Establishing Proof of Concept for Targeting IL-23 in Crohn's Disease

The potential therapeutic role of IL-23 in Crohn's disease was first established by clinical studies of IL-12/23p40 antagonists (briakinumab and ustekinumab). Ustekinumab (STELARA®) was recently approved for the treatment of moderately to severely active Crohn's disease. While these programs demonstrated that blockade of both IL-12 and IL-23 is effective in treating Crohn's disease, they could not ascertain the relative contributions of the 2 cytokines.

More recent studies of 2 anti-IL-23 antagonists, risankizumab (previously BI-655066) and brazikumab (formerly MEDI2070, AMG 139), reported Phase 2 results demonstrating efficacy of IL-23 blockade in participants with moderately to severely active Crohn's disease. The magnitude of efficacy observed in each of these studies suggests the potential for improved efficacy compared with ustekinumab (anti-IL-12/23), recognizing the limitations of cross-study comparisons as well as the comparatively small size of the IL-23 Phase 2 studies.

Clinical Experience with IL-12/23-Targeted Therapy (Ustekinumab) in Crohn's Disease

The ustekinumab Phase 3 program in Crohn's disease included two 8-week studies evaluating the efficacy and safety of ustekinumab intravenous (IV) induction, and one maintenance study evaluating the efficacy and safety of ustekinumab subcutaneous (SC) maintenance, for a total duration of 52 weeks of treatment. Ustekinumab was evaluated in the full spectrum of biologic-eligible patients with Crohn's disease, ie, those who were conventional therapy failures and those who were biologic therapy failures. After a single ustekinumab ˜6 mg/kg IV induction dose at Week 0, approximately 21% and 40% of BIO-failure and CON-failure participants, respectively (versus approximately 7% and 20% of placebo-treated participants, respectively), achieved clinical remission at Week 8 (as evaluated by the Crohn's Disease Activity Index [CDAI]). Among participants who responded to ustekinumab IV induction and were rerandomized to receive ustekinumab SC maintenance 90 mg every 8 weeks (q8w) or 90 mg every 12 weeks (q12w), approximately 53% and 49% of participants were in clinical remission at Week 52, respectively, compared with 36% of participants who received placebo maintenance.

Clinical Experience with IL-23-Targeted Therapy in Crohn's Disease

Recent Phase 2 studies of 2 IL-23 mAbs, risankizumab and brazikumab, demonstrated their efficacy in improving clinical signs and symptoms, reducing inflammatory biomarkers, and improving endoscopic findings in participants primarily with biologic-refractory Crohn's disease.

Cross-study comparisons of clinical remission rates with the TL-23 blockers suggest the potential for improved efficacy compared with ustekinumab. It is notable that the induction doses used in the studies of both risankizumab (200 and 600 mg IV at Weeks 0, 4, 8) and brazikumab (700 mg IV at Weeks 0, 4) were considerably higher than approved ustekinumab dosing (˜6 mg/kg IV at Week 0). A cross-compound meta-analysis suggests that the risankizumab dosing, in particular, may be at the higher end of the dose-response curve.

Furthermore, the Phase 2 study with risankizumab also suggested the potential that response rates may not reach maximum until after 6 months of treatment. With doses of 600 mg IV every 4 weeks (q4w) for up to 6 months, clinical remission rates of approximately 50% were observed in all-treated patients, substantially higher than remission rates previously reported for other agents, including ustekinumab, in similar study populations at similar follow-up time points. Of those participants who were in remission at 6 months and who continued risankizumab maintenance treatment (180 mg SC q8w), approximately 70% were in remission at 1 year.

### Overall Rationale for Guselkumab in Crohn's Disease

In summary, the collective genetic and preclinical evidence implicates the prominent role of selectively targeting IL-23 in modulating the underlying pathophysiology of IBD. The available clinical experience of 2 IL-23 antagonists and the established evidence from an approved IL-12/23 antagonist (ustekinumab) have demonstrated proof of mechanism and proof of concept, respectively, for targeting IL-23 in the treatment of Crohn's disease. Together, the available evidence provide support for investigating guselkumab in the treatment of Crohn's disease.

### Primary Endpoint

The primary endpoint is clinical remission at Week 12 (defined as CDAI score<150). For this endpoint, comparisons of each guselkumab group with placebo will be made.

### Major Secondary Endpoints

The major secondary endpoints are described below.
Clinical remission at Week 48 (defined as CDAI<150)
Durable clinical remission at Week 48 (defined as CDAI<150 for ≥80% of all visits between Week 12 and Week 48 [ie, at least 8 of 10 visits], which must include Week 48)
Corticosteroid-free clinical remission at Week 48 (defined as CDAI score<150 at Week 48 and not receiving corticosteroids at Week 48)
PRO-2 remission at Week 12 (defined as an AP mean daily score at or below 1 AND SF mean daily score at or below 3, ie, AP≤1 and SF≤3)
PRO-2 remission at Week 48
Endoscopic response at Week 12 (defined as at least 50% improvement from baseline in SES-CD score or SES-CD score ≤2)
Endoscopic response at Week 48
Fatigue response at Week 12 (based on the PROMIS Fatigue Short Form 7a; to be defined in the SAP)

The short-term endpoints at Week 12 will be based on comparisons of each guselkumab group with the placebo group, and the long-term endpoints at Week 48 will be based on comparisons of each guselkumab group with the ustekinumab group.

From a nonclinical perspective, the risk to Crohn's disease patients is considered low when guselkumab is administered IV once every 4 weeks at doses up to 1200 mg (approximately 16 mg/kg in humans) followed by the proposed maintenance doses of up to 200 mg SC q4w, based on no adverse findings observed in cynomolgus monkeys following 5 weeks of once-weekly subchronic IV dosing at 50 mg/kg and 24 weeks of chronic once-weekly SC dosing. As summarized above, the actual exposure data (area under the serum concentration versus time curve [AUC]) achieved in monkeys relative to the predicted Week 8 to Week 12 IV clinical induction dosing interval AUC, or steady-state SC maintenance interval AUC (both normalized to weekly dosing to compare with the monkey dosing interval) provide ample exposure margins for the proposed clinical doses. This is further supported by the fact that guselkumab is a late-stage biotherapeutic with a good clinical safety profile in participants with plaque psoriasis, with data generated primarily at 100 mg SC, but also at doses up to 300 mg SC and 10 mg/kg IV in a limited number of patients with plaque psoriasis and in healthy normal volunteers, respectively, during Phase 1 of clinical development. Lastly, risankizumab (an IL-23 inhibitor with clinical potency comparable to guselkumab) has been studied in patients with Crohn's disease at up to 600 mg IV given q4w for 6 months and was reported to be well-tolerated.

Guselkumab has undergone extensive nonclinical and clinical development. The collective efficacy and safety results of the Phase 1, Phase 2, and Phase 3 clinical studies in healthy volunteers and patients with plaque psoriasis and the recent regulatory approval for the plaque psoriasis indication established a favorable benefit-risk profile for guselkumab in the treatment of plaque psoriasis. This clinical experience provided support to the ongoing development of guselkumab in other inflammatory diseases such as PsA, GPP, EP, and PPP.

Available animal and human data support the critical role of IL-23 in the pathogenesis of Crohn's disease, and studies with other anti-IL-23 mAbs suggest that selective targeting of IL-23 may achieve higher levels of efficacy than that observed with other mechanisms of action, including ustekinumab, in patients with moderately to severely active Crohn's disease.

Clinical data with ustekinumab and other anti-IL-23 mAbs suggest that maximum efficacy in Crohn's disease may require higher doses and exposures than those used in psoriasis. For example, initial dosing of ustekinumab in Crohn's disease (˜6 mg/kg IV in a 70 kg patient) is approximately 4-fold higher than in psoriasis (45 mg SC at Week 0 and Week 4). Therefore, induction doses up to 1200 mg IV given q4w for 3 doses and maintenance doses up to 200 mg SC q4w will be studied in the Phase 2 portion of this trial to evaluate whether higher doses and exposures of guselkumab are needed for maximum efficacy in Crohn's disease. Data from non-clinical toxicology studies provide adequate exposure margins for the proposed clinical doses in this protocol. In addition, comparable doses/exposures have been previously evaluated in the Phase 2 studies of 2 other anti-IL-23 mAbs, and no significant safety concerns have been reported after treatment through 1 year.

The approved dose regimen of guselkumab in psoriasis (100 mg SC at Week 0 and Week 4, and then q8w) has been demonstrated to have a favorable safety profile, and dose regimens as high as 200 mg SC q8w have been shown to have favorable safety in a Phase 2 trial in rheumatoid arthritis. The main risk is infection. Other potential safety concerns, also described in greater detail in the guselkumab IB, are based on guselkumab being an immunomodulatory mAb and include malignancy and hypersensitivity. Since the higher dose regimens of guselkumab (as proposed in this protocol) have not been previously studied, safety will be evaluated in an initial cohort of 25 patients by an independent Data Monitoring Committee (DMC).

The early safety evaluation of the initial cohort will ensure acceptable safety for continued study of the proposed Phase 2 and Phase 3 dose regimens in larger numbers of patients, and the ongoing unblinded safety assessments by the DMC throughout the Phase 2 and 3 studies will ensure patient safety in the overall development program.

### Active Comparator: Ustekinumab

Ustekinumab (STELARA) is the active comparator in this protocol. Ustekinumab is a human IgG1 kappa mAb that binds with high affinity and specificity to the p40 subunit common to both human IL-12 and human IL-23. Ustekinumab is an approved treatment for moderately to severely active Crohn's disease in adult patients in several countries including the US, Canada, and the EU; submissions for regulatory approval of the Crohn's disease indication are currently under review in a number of countries globally. The proposed induction and maintenance dosing of ustekinumab in this protocol is consistent with the currently approved country labels globally, and is consistent with the dose regimens evaluated in the ustekinumab Phase 3 clinical development program in Crohn's disease that established the efficacy and safety of ustekinumab in patients with moderately to severely active Crohn's disease.

### Phase 2 Dose-Ranging Study (GALAXI 1)

### Objectives

### Primary Objectives

To evaluate the clinical efficacy of guselkumab in participants with Crohn's disease
To evaluate the safety of guselkumab

### Secondary Objectives

To evaluate the dose-response of guselkumab to inform dose selection for the Phase 3 portion of this protocol
To evaluate the efficacy of guselkumab on endoscopic improvement
To evaluate the pharmacokinetics (PK), immunogenicity, and pharmacodynamics (PD) of guselkumab therapy, including changes in C-reactive protein (CRP) and fecal calprotectin

### Other Objectives

To evaluate the impact of guselkumab on health-related quality of life (HRQOL) and health economics outcome measures
To evaluate the efficacy of guselkumab on histologic improvement
To evaluate the impact of treatment with guselkumab on intestinal mucosal gene expression profiles and cellular composition associated with Crohn's disease

### Endpoints

The primary endpoint and major secondary endpoints evaluate the short-term efficacy of guselkumab versus placebo. These endpoints are described below.

### Primary Endpoint

Change from baseline in the CDAI score at Week 12.

### Major Secondary Endpoints

Clinical remission at Week 12 (defined as CDAI score<150).
Clinical response at Week 12 (defined as ≥100-point reduction from baseline in CDAI score or CDAI score<150).
PRO-2 remission at Week 12 (defined as an abdominal pain [AP] mean daily score at or below 1 AND stool frequency [SF] mean daily score at or below 3, ie, AP≤1 and SF≤3).
Clinical-biomarker response at Week 12 (clinical response based on CDAI score and >50% reduction from baseline in CRP or fecal calprotectin).
Endoscopic response at Week 12 (defined as at least 50% improvement from baseline in the Simple Endoscopic Score for Crohn's Disease [SES-CD] or SES-CD score ≤2)

### Hypothesis

The primary hypothesis for GALAXI 1 is that guselkumab is superior to placebo in inducing a reduction from baseline in CDAI score in participants with moderately to severely active Crohn's disease.

### Phase 3 Dose-Confirming Studies (GALAXI 2 and GALAXI 3)

GALAXI 2 and GALAXI 3 are identical studies and have the same objectives and endpoints, as

### Objectives

### Primary Objectives

To evaluate the clinical efficacy of guselkumab in participants with Crohn's disease
To evaluate the safety of guselkumab

### Secondary Objectives

To evaluate the efficacy of guselkumab on endoscopic improvement
To evaluate the impact of guselkumab on HRQOL
To evaluate the PK, immunogenicity, and PD of guselkumab therapy, including changes in CRP and fecal calprotectin

### Other Objectives

To evaluate the impact of guselkumab on health economics outcome measures
To evaluate the efficacy of guselkumab on histologic improvement
To evaluate the impact of treatment with guselkumab on intestinal mucosal gene expression profiles and cellular composition associated with Crohn's disease

### Endpoints

### Primary Endpoint

The primary endpoint is clinical remission at Week 12 (defined as CDAI score<150). For this endpoint, comparisons of each guselkumab group with placebo will be made.

### Major Secondary Endpoints

The major secondary endpoints are described below.
Clinical remission at Week 48 (defined as CDAI<150)
Durable clinical remission at Week 48 (defined as CDAI<150 for ≥80% of all visits between Week 12 and Week 48 [ie, at least 8 of 10 visits], which must include Week 48)
Corticosteroid-free clinical remission at Week 48 (defined as CDAI score<150 at Week 48 and not receiving corticosteroids at Week 48)
PRO-2 remission at Week 12 (defined as an AP mean daily score at or below 1 AND SF mean daily score at or below 3, ie, AP≤1 and SF≤3)
PRO-2 remission at Week 48
Endoscopic response at Week 12 (defined as at least 50% improvement from baseline in SES-CD score or SES-CD score ≤2)
Endoscopic response at Week 48
Fatigue response at Week 12 (based on the PROMIS Fatigue Short Form 7a; to be defined in the SAP)

The short-term endpoints at Week 12 will be based on comparisons of each guselkumab group with the placebo group, and the long-term endpoints at Week 48 will be based on comparisons of each guselkumab group with the ustekinumab group.

### Hypothesis

The primary hypothesis for both GALAXI 2 and GALAXI 3 is that guselkumab is superior to placebo in achieving clinical remission at Week 12 in participants with moderately to severely active Crohn's disease.

GALAXI 2 and GALAXI 3 will also evaluate the relative performance of long-term treatment with guselkumab versus ustekinumab. For the major secondary hypotheses for comparison with ustekinumab, while the ultimate goal is to demonstrate that the efficacy of guselkumab is superior to ustekinumab, an initial test for non-inferiority will also be performed because the overall profile of guselkumab may be favorable compared with ustekinumab (in terms of overall efficacy and safety), even if final results only indicate the relative efficacy is non-inferior to ustekinumab for a certain endpoint.

### Study Design

### Overall Design

The clinical development program for guselkumab in Crohn's disease will be conducted under this single protocol: a Phase 2/3, randomized, double-blind, placebo- and active-controlled (ustekinumab), parallel-group, multicenter protocol to evaluate the safety and efficacy of guselkumab in participants with moderately to severely active Crohn's disease who have demonstrated an inadequate response or failure to tolerate previous conventional therapy or biologic therapy.

An overview of this clinical development program is described briefly below. Under this protocol, there are 3 separate studies: a 48-week Phase 2 dose-ranging study (ie, GALAXI 1) and 2 identical 48-week Phase 3 confirmatory studies (ie, GALAXI 2 and GALAXI 3). All 3 studies will be conducted using a treat-through study design, ie, participants are randomized to treatment regimens at Week 0 and will remain on that treatment regimen through at least Week 48 of each study, unless otherwise indicated.

In the Phase 2 dose-ranging study (ie, GALAXI 1), the safety and efficacy of guselkumab dose regimens spanning a wide induction and maintenance dose range will be evaluated to support the selection of induction and maintenance dose regimens for confirmatory evaluation in Phase 3. It is estimated that 250 to 500 participants may be required to select the dose regimens that will be evaluated in Phase 3 (GALAXI 2 and GALAXI 3). Therefore, the first 250 participants in GALAXI 1 will be enrolled into an Initial Dose Decision Cohort; an interim analysis (IA) primarily based on this cohort will occur once these participants reach Week 12 (or terminate study participation prior to Week 12). Since data from more participants may be required to inform the dose decision, enrollment will continue and newly enrolled participants (i.e., starting from participant #251) will be randomized into a Transition Cohort while data from the Initial Dose Decision Cohort are being collected and analyzed. The purpose of the Transition Cohort will be to continue accruing safety and efficacy data on the Phase 2 dose regimens without interrupting the study, thereby increasing the size of the overall safety database as well as possibly contributing additional information in making a dose decision should there be uncertainty on dose selection based on the results from the Initial Dose Decision Cohort. It is anticipated that up to 500 participants will be enrolled into GALAXI 1 (ie, 250 in the Initial Dose Decision Cohort and up to 250 in the Transition Cohort) prior to the dose decision. If a dose decision for Phase 3 is not made by the time the 500th patient is randomized, enrollment will be paused until a decision for Phase 3 dosing, or a decision to terminate the development program, is made.

This is an operationally seamless protocol, ie, there will be no break in enrollment between the Phase 2 and Phase 3 studies if a dose decision can be made before 500 patients are randomized. Transition from the Phase 2 portion to the Phase 3 portion of the protocol will occur once the dose decision for Phase 3 has been made and implemented. All participants randomized after the dose decision has been implemented will be part of the Phase 3 studies.

In the Phase 3 dose-confirming studies (i.e., GALAXI 2 and GALAXI 3), the safety and efficacy of the selected guselkumab dose regimens will be evaluated. A target of 770 participants will be enrolled in each of the Phase 3 studies, for a total target sample size of 1,540 participants in the Phase 3 portion of the protocol.

Participants who complete the 48-week Phase 2 or Phase 3 studies may be eligible to enter the LTE to receive approximately 2 additional years of treatment.

The overall GALAXI Phase 2/3 protocol will enroll a total of approximately 2,000 participants, with a total duration for each participant of up to approximately 3 years.

### Target Population

The target population in all 3 studies under this protocol will be identical and will consist of men or women ≥18 years of age at the time of informed consent with moderately to severely active Crohn's disease (of at least 3 months' duration). Participants must have colitis, ileitis, or ileocolitis previously confirmed by radiography, histology, and/or endoscopy.

### Active Disease Criteria

At baseline, participants must have active Crohn's disease, defined as follows:

### Clinically Active Crohn's Disease

a. CDAI score ≥220 but ≤450
AND EITHER
b. Mean daily SF count >3, based on the unweighted CDAI component of the number of liquid or very soft stools
OR
c. Mean daily AP score >1, based on the unweighted CDAI component of abdominal pain
AND

### 2. Endoscopic Evidence of Ileocolonic Crohn's Disease

A SES-CD score ≥3, as assessed by central endoscopy reading at the screening endoscopy, which indicates the presence of at least one large ulcer (in the ileum, colon, or both) that results in:
a. a minimum score of 2 for the component of “size of ulcers”
AND
b. a minimum score of 1 for the component of “ulcerated surface”.

Within each of the studies, a maximum of 10% of the total enrolled population will be participants who have baseline scores for SES-CD<4 (ie, for participants with isolated ileal disease), or SES-CD<7 (ie, for participants with colonic or ileocolonic disease).

### Medication History Criteria

In addition, a broad participant population eligible for systemic therapy will be evaluated in this protocol and will include participants who have demonstrated an inadequate response or failed to tolerate previous conventional therapy or biologic therapy.

Note that participants with prior exposure to IL-12/23 or IL-23 agents are ineligible for entry into this protocol, with the exception of participants who have had limited exposure to and who have not demonstrated failure or intolerance to ustekinumab.

### Conventional Therapy Failure or Intolerance (CON-Failure)

Participants must have demonstrated an inadequate response to, or have failed to tolerate, at least 1 of the following conventional Crohn's disease therapies: oral corticosteroids (including prednisone, budesonide, and beclomethasone dipropionate) or the immunomodulators azathioprine (AZA), 6-mercaptopurine (6-MP) or methotrexate (MTX). Participants who have demonstrated corticosteroid dependence (ie, an inability to successfully taper corticosteroids without a return of the symptoms of Crohn's disease) are also eligible. Participants may be naïve to biologic therapy (ie, a TNF antagonist or vedolizumab or ustekinumab) or may have been exposed to biologic therapy but have not demonstrated inadequate response or intolerance. Within each of the studies, a minimum of 25% and a maximum of 50% of the total enrolled population will be participants who are CON-Failures.

### Biologic Therapy Failure or Intolerance (BIO-Failure)

Participants must have demonstrated an inadequate response to, or have failed to tolerate, at least 1 or more biologic therapies (ie, TNF antagonists or vedolizumab) at a dose approved for the treatment of Crohn's disease. Inadequate response is defined as: Primary non-response (i.e., no initial response) or Secondary non-response (i.e., response initially but subsequently lost response). Participants who have demonstrated an inadequate response to, or have failed to tolerate ustekinumab are not eligible.

The use of concomitant and prohibited therapies is described below. In general, concomitant therapies should maintain stable dosing (except for steroid tapering) and new concomitant therapies should not be initiated unless considered medically necessary by the investigator. Corticosteroids will be tapered beginning at Week 12. Initiation of prohibited therapies will result in study intervention discontinuation (SID). Finally, in the event of persistent inadequate response or clinically significant Crohn's disease worsening, discontinuation of study intervention should be strongly considered.

### Evaluations

Throughout the 3 studies, efficacy, PK, biomarkers, and safety will be assessed at time points indicated in the appropriate Schedule of Activities.

A pharmacogenomic blood sample will be collected from participants who consent to this component of the protocol (where local regulations permit). Participation in pharmacogenomic research is optional. Deoxyribonucleic acid (DNA) samples will be analyzed for identification of genetic factors that may be associated with clinical response.

An external independent DMC, with defined roles and responsibilities as governed by a DMC charter, will assess the safety of participants across the 3 studies. The DMC's initial responsibility will be careful review of the safety data from the first 25 participants randomized and treated in GALAXI 1. After that, ongoing safety data reviews will continue as specified in the DMC charter. After each review, the DMC will make recommendations to the sponsor about the continuation of the studies.

### Phase 2 Dose-Ranging Study (GALAXI 1)

### Overview of Phase 2 Study Design and Dose Decision for Phase 3

At Week 0, participants will be randomized in a 1:1:1:1:1 ratio to receive 1 of 3 dose regimens of guselkumab, ustekinumab, or placebo. Participants will be allocated to a treatment group using a permuted block randomization with baseline CDAI score (≤300 or >300) and prior BIG-Failure status (Yes/No) as the stratification variables. A minimum of 25% and a maximum of 50% of the total enrolled population will be CON-Failure participants. In addition, a maximum of 10% of the total enrolled population will have baseline scores for SES-CD<4 (ie, for participants with isolated ileal disease), or SES-CD<7 (ie, for participants with colonic or ileocolonic disease). Allocation to treatment group will be performed using a central randomization center by means of an interactive web response system (IWRS).

It is anticipated that up to 500 participants will be enrolled into GALAXI 1 (i.e., 250 in the Initial Dose Decision Cohort and up to 250 in the Transition Cohort) prior to the dose decision for Phase 3. If a dose decision for Phase 3 is not made by the time the 500th patient is randomized, enrollment will be paused until a decision for Phase 3 dosing, or a decision to terminate the development program, is made.

Interim analyses are planned at Week 12 (and at Week 24, if necessary) after all participants from the Initial Dose Decision Cohort have either completed the Week 12 (or Week 24) visit or terminated study participation prior to the Week 12 (or Week 24) visit to inform the dose decision for Phase 3. At the time of each IA, all available data from both the Initial Dose Decision Cohort and the Transition Cohort will be analyzed, including any data beyond Week 12. Additional data transfers and analyses may be performed at other time points if needed to enable the dose decision for Phase 3. The goal is to select 2 guselkumab dose regimens for confirmatory evaluation in Phase 3.

### Treatment Groups

An overview of the 5 treatment groups and their corresponding dosing schemes from Week 0 through Week 48 of the Phase 2 study is provided below.

Dosing Schemes for the 5 Treatment Groups from Week 0 to Week 48 in Phase 2 (Ie, GALAXI 1)

All participants in the Phase 2 study (i.e., Initial Dose Decision Cohort and Transition Cohort) will be randomized to 1 of 5 treatment groups as described below. Participants will remain on their assigned treatment regimens through the end of the 48-week study, except for the Placebo group as outlined below.

Group 1: Guselkumab Regimen 1 (1200 mg IV q4w×3→200 mg SC q4w)

Participants will receive guselkumab 1200 mg IV induction q4w from Week 0 through Week 8 (i.e., total of 3 IV doses). At Week 12, participants will continue treatment with guselkumab 200 mg SC maintenance q4w through Week 44.

Group 2: Guselkumab Regimen 2 (600 mg IV q4w×3→200 mg SC q4w)

Participants will receive guselkumab 600 mg IV induction q4w from Week 0 through Week 8 (ie, total of 3 IV doses). At Week 12, participants will continue treatment with guselkumab 200 mg SC maintenance q4w through Week 44.

Group 3: Guselkumab Regimen 3 (200 mg IV q4w×3→100 mg SC q8w)

Participants will receive guselkumab 200 mg IV induction q4w from Week 0 through Week 8 (ie, total of 3 IV doses). At Week 16, participants will continue treatment with guselkumab 100 mg SC maintenance q8w through Week 40.

Group 4: Active Control, Ustekinumab (˜6 mg/kg IV→90 mg SC q8w)

Participants will receive a single ustekinumab IV induction dose at Week 0 (weight-based IV doses approximating 6 mg/kg as outlined below). At Week 8, participants will receive ustekinumab SC maintenance (90 mg SC q8w) through Week 40.
Ustekinumab 260 mg (weight ≤55 kg)
Ustekinumab 390 mg (weight >55 kg and ≤85 kg)
Ustekinumab 520 mg (weight >85 kg)

### Group 5: Placebo→Placebo or Ustekinumab Crossover

Participants will receive placebo IV q4w from Week 0 through Week 8 (ie, total of 3 IV doses). At Week 12, participants will continue treatment based on their clinical response status as follows:
Placebo responders: Continue placebo treatment q4w from Week 12 through Week 44.
Placebo nonresponders: Receive a single ustekinumab IV induction dose at Week 12 (weight-based IV doses approximating 6 mg/kg as outlined above). At Week 20, participants will receive ustekinumab SC maintenance (90 mg SC q8w) through Week 44.

Clinical response is defined as a reduction from baseline (ie, Week 0) in the CDAI score of ≥100 points or being in clinical remission (CDAI<150). To maintain the blind, participants in all treatment groups will be assessed for their clinical response status at Week 12. In addition, placebo administrations (IV and SC) will be given, as appropriate, to maintain the blind throughout the duration of the study. No dosing adjustments are planned for any of the treatment groups from Week 0 through Week 48, except for Group 5 (Placebo) at Week 12 based on clinical response status as described above.

The use of concomitant and prohibited therapies is described below. In general, concomitant therapies should maintain stable dosing (except for steroid tapering) and new concomitant therapies should not be initiated, unless considered medically necessary by the investigator. Corticosteroids will be tapered beginning at Week 12. Initiation of prohibited therapies will result in SID. Finally, in the event of persistent inadequate response or clinically significant Crohn's disease worsening, discontinuation of study intervention should be strongly considered.

All participants who complete the Week 48 evaluations may be eligible to enter the LTE and continue to receive study intervention for approximately 2 additional years (Week 48 to Week 156).

### Endpoints and Evaluations

The primary endpoint is change from baseline in the CDAI score at Week 12. The major secondary endpoints are: clinical remission at Week 12, clinical response at Week 12, PRO-2 remission at Week 12, endoscopic response at Week 12, and clinical-biomarker response at Week 12. Analyses of these endpoints will be based on comparisons between each guselkumab group and the placebo group. Additional analyses of endpoints at other time points, including comparisons of guselkumab with ustekinumab at Week 48, will also be performed.
Efficacy, PK, and PD parameters, biomarkers, and safety will be assessed.
Database locks (DBLs) are planned for Week 12 and Week 48. Additional DBLs (e.g., Week 24) may be added as necessary.

### Phase 3 Dose-Confirming Studies (GALAXI 2 and GALAXI 3)

### Overview of Phase 3 Design

At Week 0, a target of 1,540 participants will be randomly allocated to GALAXI 2 (n=770) or GALAXI 3 (n=770), using a permuted block randomization with baseline CDAI score (≤300 or >300), baseline SES-CD score (≤12 or >12), prior BIO-Failure status (Yes/No), and baseline corticosteroid use (Yes/No) as the stratification variables. Within each stratum, participants in each study will be randomized in a 2:2:2:1 ratio to receive 1 of 2 dose regimens of guselkumab, ustekinumab, or placebo. Within each study (GALAXI 2 and GALAXI 3), a minimum of 25% and a maximum of 50% of the total enrolled population will be participants who are CON-Failures. In addition, a maximum of 10% of the total enrolled population will have baseline scores for SES-CD<4 (ie, for participants with isolated ileal disease) or SES-CD<7 (ie, for participants with colonic or ileocolonic disease). Allocation to treatment groups will be performed using a central randomization center by means of an IWRS.

### Groups

The Phase 3 guselkumab dose regimens will be selected based on the efficacy and safety of the induction dose range (i.e., from 200 mg to 1200 mg IV) and maintenance dose range (i.e., from 100 mg SC q8w to 200 SC q4w) evaluated in the Phase 2 study.

Based on the Phase 2 data, 2 guselkumab dose regimens (i.e., IV induction→SC maintenance) will have been selected for confirmatory evaluation in Phase 3. Identical dose regimens are to be evaluated in both Phase 3 studies.

An overview of the 4 treatment groups in the 2 Phase 3 studies and their corresponding dosing schemes from Week 0 through Week 48 are summarized below. Participants will remain on their assigned treatment regimens through the end of the 48-week study, except for the Placebo group as outlined below.

Dosing Schemes for the 4 Treatment Groups from Week 0 to Week 48 in the Phase 3 Studies (i.e., GALAXI 2 and GALAXI 3)

### Group 1 and Group 2: Guselkumab Regimen 1 and Guselkumab Regimen 2

Participants will receive guselkumab IV induction q4w from Week 0 through Week 8 (i.e., total of 3 IV doses). Depending on whether the selected SC maintenance dose is given q4w and/or q8w, participants will continue treatment with guselkumab SC maintenance starting at Week 12 through Week 44 (i.e., if q4w regimen) or starting at Week 16 through Week 40 (i.e., if q8w regimen).

Group 3: Active Control—Ustekinumab (−6 mg/kg IV→90 mg SC q8w)

Participants will receive a single ustekinumab IV induction dose at Week 0 (weight-based IV dose approximating 6 mg/kg as outlined below). At Week 8, participants will receive ustekinumab SC maintenance (90 mg SC q8w) through Week 40.
Ustekinumab 260 mg (weight ≤55 kg)
Ustekinumab 390 mg (weight >55 kg and ≤85 kg)
Ustekinumab 520 mg (weight >85 kg)

### Group 4: Placebo→Placebo or Ustekinumab Crossover

Participants will receive placebo IV q4w from Week 0 through Week 8 (i.e., total of 3 IV doses). At Week 12, participants will continue treatment based on their clinical response status as follows:
Placebo responders: Continue placebo treatment from Week 12 through Week 44.
Placebo nonresponders: Receive a single ustekinumab IV induction dose at Week 12 (weight-based IV doses approximating 6 mg/kg as outlined above). At Week 20, participants will receive ustekinumab SC maintenance (90 mg SC q8w) through Week 44.

Clinical response is defined as a reduction from baseline (ie, Week 0) in the CDAI score of ≥100 points or being in clinical remission (CDAI<150). To maintain the blind, participants in all treatment groups will be assessed for their clinical response status at Week 12.

In addition, placebo administrations (IV and SC) will be given, as appropriate, to maintain the blind throughout the duration of the study. No dosing adjustments are planned for any of the treatment groups from Week 0 through Week 48, except for Group 4 (Placebo) at Week 12 based on clinical response status as described above.

The use of concomitant and prohibited therapies is described below. In general, concomitant therapies should maintain stable dosing (except for steroid tapering) and new concomitant therapies should not be initiated; unless considered medically necessary by the investigator. Corticosteroids will be tapered beginning at Week 12. Initiation of prohibited therapies will result in SID. Finally, in the event of persistent inadequate response or clinically significant Crohn's disease worsening, discontinuation of study intervention should be strongly considered.

All participants who complete the Week 48 evaluations may be eligible to enter the LTE and continue to receive approximately 2 additional years of treatment.

### Endpoints and Evaluations

Both GALAXI 2 and GALAXI 3 have the same primary and major secondary endpoints.

The primary endpoint is clinical remission at Week 12, based on comparisons between guselkumab and placebo. The major secondary endpoints of clinical remission at Week 48, durable clinical remission at Week 48, corticosteroid-free clinical remission at Week 48, PRO-2 remission at Week 48, and endoscopic response at Week 48 are based on comparisons between guselkumab and ustekinumab. The major secondary endpoints of PRO-2 remission at Week 12, endoscopic response at Week 12, and fatigue response at Week 12 are based on comparisons between each guselkumab treatment group and the placebo group.

Efficacy, PK, and PD parameters, biomarkers, and safety will be assessed.
A DBL is planned for Week 48. Additional DBLs may be added if necessary and will be specified in the SAP.

### Long-Term Extension

The LTE will be conducted for approximately 2 years, from Week 48 through Week 156.

At Week 48 of GALAXI 1, GALAXI 2, or GALAXI 3, all participants who, in the opinion of the investigator, will continue to benefit from treatment (i.e., based on Week 48 clinical and endoscopic evaluations) are eligible to enter the LTE to receive approximately 2 additional years of treatment, during which time the longer-term efficacy and safety of guselkumab will be evaluated. All participants will be assessed. The final efficacy and safety follow-up (FES) visit of the LTE will occur at approximately Week 156 (i.e., approximately 16 weeks after their last study intervention administration at Week 140).

Participants who are not eligible to enter the LTE at Week 48 are to return for a FES visit 16 weeks after their last study intervention administration.

During the LTE, all participants will continue to receive the same treatment regimen (ie, guselkumab, ustekinumab, or placebo) that they were receiving at the end of GALAXI 1, GALAXI 2, or GALAXI 3. The first study intervention administration in the LTE will occur at Week 48 and the last study intervention administration will occur at Week 140. Treatment adjustment for inadequate response is permitted between Week 52 and Week 80 of the LTE.

Beginning at Week 48, at the discretion of the investigator and participant, and after appropriate and documented training, participants may self-administer study intervention at the investigative site. A caregiver may also be trained to administer study intervention. After receiving training at Week 48, participants who are eligible for self- (or caregiver) administration of study intervention will be supplied with study intervention for at-home administration and will have their first at-home administration at Week 52. Participants who are unable or unwilling to have study intervention administered away from the investigative site will continue administration at the investigative site.

All participants will continue to receive active or placebo study intervention administration in the LTE in a blinded fashion until study unblinding, which will occur after the Week 48 DBL and the Week 48 analyses have been completed for the Phase 2 study (for participants entering the LTE from GALAXI 1) or for the Phase 3 studies (for participants entering the LTE from GALAXI 2 or GALAXI 3).

After study unblinding, all participants who are on active treatment (i.e., guselkumab or ustekinumab) will continue to receive their assigned active treatment for the remaining duration of the LTE through Week 140. Participants who are on placebo will be discontinued from study intervention upon study unblinding, and will have an FES visit at that time.

### Treatment Adjustment for Inadequate Response

Participants from all treatment groups (i.e., guselkumab, ustekinumab, and placebo) who meet inadequate response criteria between Week 52 (i.e., the first visit at which treatment adjustment is permitted) and Week 80 (i.e., the last visit at which treatment adjustment is permitted) will be eligible for a single treatment adjustment (i.e., the first-time inadequate response criteria are met).

Inadequate response is defined as not being in clinical response AND having a CDAI score of at least 220 points. Clinical response is defined as a reduction from baseline (ie, Week 0) in the CDAI score of ≥100 points or being in clinical remission (CDAI<150).

Participants (who are receiving placebo, ustekinumab, or the lower SC maintenance dose of guselkumab) will be eligible to receive a single, blinded, treatment adjustment to the highest guselkumab SC maintenance dose as defined in the Phase 2 or the Phase 3 portion of the protocol in which they are enrolled. Participants who are already receiving the highest guselkumab SC maintenance dose will receive a single, blinded, sham treatment adjustment. Participants who have received treatment adjustments will remain on their new treatment regimen through Week 92.

At Week 96, the benefit of treatment adjustment will be evaluated. Continued participation in the remaining duration of the LTE will be decided on investigator's clinical judgment of the results of the Week 96 clinical and endoscopic evaluations. Discontinuation of study intervention should be considered in participants with persistent unsatisfactory response or clinically significant worsening Crohn's disease where continuation of the study intervention is not in the best interest of the participant.

### Endpoints and Evaluations

Through Week 156, the longer-term efficacy and safety of guselkumab will be evaluated. In addition, the benefit of treatment adjustment will be evaluated based on descriptive analysis of various efficacy endpoints (to be specified in the SAP).

Database locks are planned at Week 96 and when the final participant has completed the final efficacy and safety visit in the LTE. Additional DBLs may be added if necessary, and will be specified in the Phase 3 SAP.

### Use of Placebo- and Active-Control

The inclusion of both placebo and active controls in the same protocol has several advantages. A short-term placebo-control period facilitates the evaluation of the short-term efficacy and safety of a new treatment compared with placebo within a timeframe for which the use of placebo in participants with active disease is considered clinically acceptable in support of scientific research. For longer-term treatment, the use of an active comparator control can alleviate the concern over the extended use of placebo and can also provide an opportunity to evaluate comparative efficacy and safety in a randomized-controlled setting. There is significant clinical value to determine whether a new treatment option will provide similar or greater benefit to patients compared with an approved treatment option.

Ustekinumab was selected as the active comparator because it targets an overlapping mechanism of action (i.e., both IL-12/23 blockade) and the preclinical evidence suggests the potential for improved efficacy with more specific targeting of IL-23. Further, the proposed dosing of ustekinumab in this protocol is the highest currently approved induction-maintenance dose regimen and was one of the dose regimens evaluated in the ustekinumab Phase 3 clinical development program in Crohn's disease. Therefore, the inclusion of ustekinumab as an active comparator in this program will provide a valuable and relevant benchmark for comparison with guselkumab.

Ustekinumab is included as an active-reference arm in the Phase 2 study to collect data that will inform treatment effect size and sample size assumptions for the Phase 3 studies. Ustekinumab is included in the 2 Phase 3 studies as an active comparator control arm to enable the randomized-controlled evaluation of the long-term efficacy and safety of the 2 guselkumab dose regimens compared with ustekinumab through approximately 1 year (i.e., Week 48) of treatment. An important objective of this development program is to determine whether the efficacy of guselkumab is superior (or, at minimum, non-inferior) to ustekinumab in achieving long-term clinical remission.

### Patient-Reported Outcomes on Health-Related Quality of Life

Patient-reported outcome (PRO) evaluations (ie, IBDQ, PROMIS-29, PROMIS Fatigue 7-item Short Form, 5-level EuroQol 5 dimensions [EQ-5D-5L] instrument) will be used to assess the benefits of guselkumab treatment on disease-specific and general HRQOL.

### Phase 2 Dose-Ranging Study (GALAXI 1)

The following guselkumab dose regimens will be evaluated through Week 48 of GALAXI 1:
Guselkumab Regimen 1—Induction: 1200 mg IV at Weeks 0, 4, 8; followed by Maintenance: 200 mg SC q4w (ie, at Weeks 12, 16, 20, 24, 28, 32, 36, 40, and 44)
Guselkumab Regimen 2—Induction: 600 mg IV at Weeks 0, 4, 8; followed by Maintenance: 200 mg SC q4w (ie, at Weeks 12, 16, 20, 24, 28, 32, 36, 40, and 44)
Guselkumab Regimen 3—Induction: 200 mg IV at Weeks 0, 4, 8; followed by Maintenance: 100 mg SC q8w (ie, at Weeks 16, 24, 32, and 40)

### Induction Dose Regimens

Cross-study comparisons between the guselkumab and risankizumab Phase 2 studies in patients with plaque psoriasis suggest that comparable efficacy was attained at almost similar dose regimens. A model-based meta-analysis also suggests comparable clinical potency for these 2 compounds. In addition, the PK of guselkumab were found to be similar to those of risankizumab.13,23 These dose-response and PK data suggest that comparable levels of IL-23 blockade and efficacy may be achieved in Crohn's disease at similar dose regimens or systemic exposures for these 2 compounds. Furthermore, a PK/PD model of ustekinumab (an IL-12/23 blocker), which is approved in Crohn's disease was considered applicable to predict efficacy following administration of different guselkumab dose regimens.

In the Phase 2 study of risankizumab in participants with moderately to severely active Crohn's disease, dose-dependent efficacy was demonstrated with a greater proportion of participants on the higher induction dose regimen of risankizumab (ie, 600 mg IV q4w) achieving remission at Week 12 compared with those receiving the lower dose regimen (ie, 200 mg IV q4w); however, it was not clear if maximum efficacy was attained with the risankizumab 600 mg IV induction dose regimen in this Phase 2 study. Dose-dependent efficacy was further demonstrated with risankizumab as shown by an increased rate of remission in patients who switched from 200 mg IV to 600 mg IV in the second period of that study (Week 12 through Week 26). Based on these findings, along with the comparable PK and clinical potency of guselkumab and risankizumab, and coupled with the PK/PD predictions of guselkumab in Crohn' disease, induction dose regimens comprising guselkumab 600 mg IV, and 200 mg IV, each given at Weeks 0, 4, and 8, were selected for the Phase 2 dose-ranging study.

Additionally, a higher dose of guselkumab (1200 mg q4w IV) induction dose regimen will evaluate the possibility of achieving a higher level of efficacy at Week 12 than that observed with the higher risankizumab dose regimen (ie, 600 mg IV) tested in Phase 2. Overall, the 3 guselkumab IV induction dose regimens provide a 6-fold range of exposure that is likely to result in adequate separation between dose levels and consequently support guselkumab induction dose selection for Phase 3.

Regarding the safety of these higher IV induction guselkumab doses, single doses of guselkumab as high as 10 mg/kg, with the highest single dose tested being 987 mg, have been previously studied in a Phase 1 plaque psoriasis study in a limited number of participants. Additionally, guselkumab IV doses of up to 50 mg/kg weekly for 5 weeks, and guselkumab SC doses of up to 50 mg/kg weekly for 24 weeks, were well-tolerated in cynomolgus monkeys and did not result in any clinical or anatomic findings. These data suggest an acceptable exposure margin between predicted guselkumab exposures for the 1200 mg IV regimen compared with those observed in toxicology studies. Furthermore, risankizumab was well-tolerated at dose regimens up to 6 doses of 600 mg IV q4w, ie, a total of 3600 mg over a period of 26 weeks. Longer-term follow-up of these participants through Week 52 did not identify any significant safety concerns based on published data. Nonetheless, an external DMC will be commissioned to monitor the benefit-risk of guselkumab.

### Maintenance Dose Regimens

The posology of other biologics in Crohn's disease suggests that once the inflammatory burden of the disease is reduced, the drug exposures required to maintain efficacy may be lower than the exposures attained with initial induction doses.

In the ustekinumab Crohn's disease Phase 3 studies, among participants who were in remission at Week 8 following an induction regimen of ˜6 mg/kg IV, a 90 mg SC q8w maintenance regimen resulted in 67% of subjects maintaining remission at Week 52. In the risankizumab Crohn's disease Phase 2 study, among participants who were in remission at Week 26 after receiving up to 6 months of 600 mg IV q4w induction dosing, the long-term uncontrolled data showed that a 180 mg SC q8w regimen resulted in 71% of patients maintaining remission at Week 52.

Accordingly, in this protocol, after 12 weeks of guselkumab IV induction treatment, dose regimens providing lower guselkumab exposures will be evaluated during SC maintenance treatment through Week 48. The selected maintenance dose regimens provide reasonable maintenance:induction exposure ratios comparable to those of other biologics approved in Crohn's disease.

Regimens 1 and 2 evaluate guselkumab 1200 mg IV q4w and 600 mg IV q4w induction, respectively. For each of these regimens, a maintenance regimen of 200 mg SC q4w will be studied to evaluate if higher exposure than that tested in the risankizumab Phase 2 study (i.e., 180 mg SC q8w) is necessary to optimize efficacy in maintenance.

For Regimen 3, which evaluates guselkumab 200 mg IV q4w induction, a maintenance regimen of 100 mg SC q8w will be studied. The guselkumab 100 mg SC q8w regimen is expected to provide efficacy at least similar to, or greater than that observed with ustekinumab 90 mg SC q8w, the maintenance dose regimen for the active comparator being evaluated in this study.

Overall, the 2 guselkumab maintenance SC dose regimens provide a 4-fold range of exposure that should support dose selection for Phase 3.

No treatment adjustments are planned for any of the treatment groups from Week 0 through Week 48 of GALAXI 1, except for IV induction placebo nonresponders who will cross over to receive the ustekinumab dose regimen being evaluated in this study (i.e., ˜6 mg/kg IV at Week 12 followed by 90 mg SC q8w from Week 20). Participants randomized to placebo IV who are responders at Week 12 will continue to receive SC placebo through Week 44.

### Phase 3 Dose-Confirming Studies (GALAXI 2 and GALAXI 3)

Based on the Phase 2 data, 2 guselkumab dose regimens (ie, IV induction→SC maintenance) will be selected for confirmatory evaluation in Phase 3.

The goal is to select a single induction dose regimen from the induction dose range evaluated (ie, 200 mg to 1200 mg IV q4w at Week 0, Week 4, and Week 8) in the Phase 2 dose-ranging study based on the totality of the efficacy, safety, and exposure-response (E-R) data at the time of dose decision. The choice of a single induction regimen to be evaluated in the Phase 3 dose-confirming studies is based on the consideration that a sufficient amount of information will be available to establish an optimal induction dose regimen. In this scenario, the selected induction dose regimen will be paired with 2 maintenance dose regimens selected from the range of exposures obtained from the guselkumab SC dose regimens evaluated in Phase 2 (i.e., between 100 mg q8w to 200 mg q4w).

It is also possible that the Phase 2 data may support the selection of more than one induction dose regimen for Phase 3 evaluation. In this case, each selected induction dose regimen will be paired with an appropriate maintenance dose regimen.

No treatment adjustments are planned for any of the treatment groups from Week 0 through Week 48 of GALAXI 2 and GALAXI 3, except for IV induction placebo nonresponders who will cross over to receive the ustekinumab dose regimen being evaluated in this study (ie, ˜6 mg/kg IV at Week 12 followed by 90 mg SC q8w from Week 20). Participants randomized to placebo IV who are responders at Week 12 will continue to receive SC placebo through Week 44.

### Long Term Extension (Week 48 to Week 144)

Participants will continue on their assigned guselkumab maintenance dose during the LTE of GALAXI 1, GALAXI 2, and GALAXI 3. Participants who experience inadequate response between Week 52 through Week 80 while on the lower of the 2 maintenance dose regimens being evaluated in the respective study will be eligible for a single dose adjustment, and will receive the higher maintenance dose until the end of the LTE to assess if they can regain clinical response.

### Inclusion Criteria

Each potential participant must satisfy all of the following criteria to be enrolled in the protocol:
1. Be male or female (according to their reproductive organs and functions assigned by chromosomal complement) ≥18 years of age.
2. Have Crohn's disease or fistulizing Crohn's disease of at least 3 months duration (defined as a minimum of 12 weeks), with colitis, ileitis, or ileocolitis, confirmed at any time in the past by radiography, histology, and/or endoscopy.
3. Have clinically active Crohn's disease, defined as a baseline CDAI score ≥220 but ≤450 and either:
a. Mean daily SF count >3, based on the unweighted CDAI component of the number of liquid or very soft stools
OR
b. Mean daily AP score >1, based on the unweighted CDAI component of abdominal pain
4. Have endoscopic evidence of active ileocolonic Crohn's disease as assessed by central endoscopy reading at the screening endoscopy, defined as a screening SES-CD score ≥3, which indicates the presence of at least 1 large ulcer (in the ileum, colon, or both) that results in:
a. a minimum score of 2 for the component of “size of ulcers”
AND
b. a minimum score of 1 for the component of “ulcerated surface”.

Within each of the studies, a maximum of 10% of the total enrolled population will be participants who have baseline scores for SES-CD<4 (i.e., for participants with isolated ileal disease) or SES-CD<7 (i.e., for participants with colonic or ileocolonic disease).
Concomitant or Previous Medical Therapies Received 5. Prior or current medication for Crohn's disease must include at least 1 of the following, and must fulfill additional criteria as described in Appendix 2 (Section 10.2), Appendix 3 (Section 10.3), and Appendix 4 (Section 10.4):
a. Current treatment with oral corticosteroids (including budesonide and beclomethasone dipropionate) and/or immunomodulators (AZA, 6-MP, MTX)
OR
b. History of failure to respond to, or tolerate, at least 1 of the following therapies: oral corticosteroids (including budesonide and beclomethasone dipropionate) or immunomodulators (AZA, 6-MP, MTX).
OR
c. History of corticosteroid dependence (ie, an inability to successfully taper corticosteroids without a return of the symptoms of Crohn's disease).
OR
d. Has previously demonstrated lack of initial response (ie, primary nonresponders), responded initially but then lost response with continued therapy (i.e., secondary nonresponders), or were intolerant to 1 or more biologic agents at a dose approved for the treatment of Crohn's disease (ie, infliximab, adalimumab, certolizumab pegol, vedolizumab, or approved biosimilars for these agents).
Note: Participants meeting criteria 5a-c may also be naïve to biologic therapy (i.e., a TNF antagonist or vedolizumab or ustekinumab) or may have been exposed to these biologic therapies but have not demonstrated inadequate response or intolerance. Participants with prior exposure to IL-12/23 or IL-23 agents are ineligible for entry into this protocol, with the exception of participants who have had limited exposure to ustekinumab at its approved labeled dosage AND have met the required wash-out criterion AND have not demonstrated failure or intolerance to ustekinumab.
6. Adhere to the following requirements for concomitant medication for the treatment of Crohn's disease. The following medications are permitted provided that doses meeting the requirements listed below are stable or have been discontinued prior to baseline within the timeframes specified below:
a. Oral 5-aminosalicylic acid (5-ASA) compounds on stable doses for at least 2 weeks; or if recently discontinued, must have been stopped for at least 2 weeks.
b. Oral corticosteroids at a prednisone-equivalent dose at or below 40 mg/day, or 9 mg/day of budesonide, or 5 mg/day beclomethasone dipropionate, and on stable dosing for at least 2 weeks; or if recently discontinued, must have been stopped for at least 2 weeks.
c. Conventional immunomodulators (i.e., AZA, 6-MP, or MTX) for at least 12 weeks and have been on a stable dose for at least 4 weeks; or if recently discontinued, must have been stopped for at least 4 weeks.
d. If receiving antibiotics as a primary treatment of Crohn's disease, doses must be stable for at least 3 weeks; or if recently discontinued, must have been stopped for at least 3 weeks.
e. If receiving enteral nutrition as a primary treatment for Crohn's disease, must have been receiving for at least 2 weeks; or if recently discontinued, must have been stopped for at least 2 weeks.

### Screening Laboratory Tests

7. Have screening laboratory test results within the following parameters, and if 1 or more of the laboratory parameters is out of range, a single retest of laboratory values is permitted during the approximately 5-week screening period:
a. Hemoglobin ≥8.0 g/dL.
b. White blood cells (WBCs) ≥3.5×103/μL.
c. Neutrophils ≥1.5×103/μL.
d. Platelets ≥100×103/μL.
e. Serum creatinine ≤1.5 mg/dL.
f. Aspartate aminotransferase (AST) and alanine aminotransferase (ALT) concentrations must be ≤2 times the upper limit of normal (ULN) range for the laboratory conducting the test.
g. Direct (conjugated) bilirubin <1.0 mg/dL.

### Tuberculosis

8. Are considered eligible according to the following tuberculosis (TB) screening criteria:
a. Have no history of latent or active TB prior to screening. An exception is made for participants who have a history of latent TB AND who satisfy one of the following criteria:
   currently receiving treatment for latent TB
   will initiate treatment for latent TB prior to or simultaneously with the first administration of study intervention
OR
   have documentation of having completed appropriate treatment for latent TB within 5 years prior to the first administration of study intervention. It is the responsibility of the investigator to verify the adequacy of previous anti-tuberculous treatment and provide appropriate documentation.
b. Have no signs or symptoms suggestive of active TB upon medical history and/or physical examination.
c. Have had no recent close contact with a person with active TB or, if there has been such contact, will be referred to a physician specializing in TB to undergo additional evaluation and, if warranted, receive appropriate treatment for latent TB prior to or simultaneously with the first administration of study intervention.
d. Within 8 weeks prior to the first administration of study intervention, have a negative QuantiFERON®-TB Gold test result, or have a newly identified positive QuantiFERON-TB Gold test in which active TB has been ruled out and for which appropriate treatment for latent TB has been initiated either prior to or simultaneously with the first study intervention administration.

Note: A negative tuberculin skin test result is additionally required if the QuantiFERON-TB Gold test is not approved/registered in the country in which this protocol is being conducted. In Ukraine, while the QuantiFERON-TB gold test is not approved/registered, it is acceptable, and an additional tuberculin skin test is not required. The QuantiFERON-TB Gold test and the tuberculin skin test are not required at screening for participants with a history of latent TB, if active TB has been ruled out, and if appropriate treatment has been initiated/completed as described above in Inclusion Criterion 8a.
e. Have a chest radiograph (both posterior-anterior and lateral views, or per country regulations where applicable), taken ≤12 weeks before the first administration of study intervention and read by a qualified radiologist, with no evidence of current, active TB or old, inactive TB.

### Contraception

Contraceptive (birth control) use by men or women should be consistent with local regulations regarding the acceptable methods of contraception for those participating in clinical studies. Typical use failure rates may differ from those when used consistently and correctly. Use should be consistent with local regulations regarding the use of contraceptive methods for participants in clinical studies.
9. A female participant of childbearing potential must have a negative urine pregnancy test result at screening and baseline.
10. Before randomization, a female participant must be:
a. Not of childbearing potential
b. Of childbearing potential and:
c. Practicing a highly effective method of contraception (failure rate of <1% per year when used consistently and correctly) and agrees to remain on a highly effective method while receiving study intervention and until 16 weeks after last dose (ie, the end of relevant systemic exposure).; however, the method selected must meet local/regional regulations/guidelines for highly effective contraception.
Note: If a participant's childbearing potential changes after start of the study (e.g., a premenarchal woman experiences menarche) or the risk of pregnancy changes (e.g., a woman who is not heterosexually active becomes active), a woman must begin using a highly effective method of contraception, as described throughout the inclusion and exclusion criteria.
11. A woman must agree not to donate eggs (ova, oocytes) for the purposes of assisted reproduction during the study and for a period of 16 weeks after the last administration of study intervention.
12. During the study and for at least 16 weeks after the last administration of study intervention, a male participant
a. who is sexually active with a female of childbearing potential must agree to use a barrier method of contraception (eg, condom with spermicidal foam/gel/film/cream/suppository).
b. who is sexually active with a pregnant female must use a condom.
c. must agree not to donate sperm for the purpose of reproduction.

### General

13. Be willing and able to adhere to the lifestyle restrictions specified in this protocol.
14. Must sign an informed consent form (ICF) indicating that he or she understands the purpose of, and procedures required for, the study and is willing to participate in the study.
15. Must sign a separate ICF if he or she agrees to provide an optional DNA sample for research (where local regulations permit). Refusal to give consent for the optional DNA research sample does not exclude a participant from participation in the study.

### 5.2. Exclusion Criteria

Any potential participant who meets any of the following criteria will be excluded from participating in the protocol:
1. Has complications of Crohn's disease, such as symptomatic strictures or stenoses, short gut syndrome, or any other manifestation, that might be anticipated to require surgery, could preclude the use of the CDAI to assess response to therapy, or would possibly confound the ability to assess the effect of treatment with guselkumab or ustekinumab.
2. Currently has or is suspected to have an abscess. Recent cutaneous and perianal abscesses are not exclusionary if drained and adequately treated at least 3 weeks before baseline, or 8 weeks before baseline for intra-abdominal abscesses, provided that there is no anticipated need for any further surgery. Participants with active fistulas may be included if there is no anticipation of a need for surgery and no abscesses are currently identified.
3. Has had any kind of bowel resection within 6 months, or any other intra-abdominal or other major surgery (eg, requiring general anesthesia) within 12 weeks, before baseline.
4. Has a draining (ie, functioning) stoma or ostomy.
5. Has a stool culture or other examination positive for an enteric pathogen, including Clostridium difficile toxin, in the previous 4 months, unless a repeat examination is negative and there are no signs of ongoing infection with that pathogen.

### Concomitant or Previous Medical Therapies Received

6. Has received any of the following prescribed medications or therapies within the specified period:
a. IV corticosteroids received within 3 weeks of baseline
b. Cyclosporine, tacrolimus, sirolimus, or mycophenolate mofetil received within 8 weeks of baseline
c. 6-thioguanine (6-TG) received within 4 weeks of baseline
d. Biologic agents:
1) Anti-TNF therapy (eg, infliximab, etanercept, certolizumab pegol, adalimumab, golimumab) received within 8 weeks of baseline
2) Vedolizumab received within 16 weeks of baseline
3) Ustekinumab received within 16 weeks of baseline
4) Other immunomodulatory biologic agents received within 12 weeks of baseline or within 5 half-lives of baseline, whichever is longer.
e. Any investigational intervention received within 4 weeks of baseline or within 5 half-lives of baseline, whichever is longer.
f. Nonautologous stem cell therapy (eg, Prochymal), natalizumab, efalizumab, or biologic agents that deplete B- or T-cells (eg, rituximab, alemtuzumab, or visilizumab) received within 12 months of baseline.
g. Treatment with apheresis (eg, Adacolumn apheresis) or total parenteral nutrition for Crohn's disease within 3 weeks of baseline.
7. Has previously received a biologic agent targeting IL-12/23 or IL-23, including but not limited to briakinumab, brazikumab, guselkumab, mirakizumab (formerly LY2525623), and risankizumab.

Exception: Participants who have had limited exposure to ustekinumab at its approved labeled dosage AND have met the required wash-out criterion AND have not demonstrated failure or intolerance to ustekinumab are not excluded from this protocol provided that other inclusion criteria have been satisfied and no other exclusion criteria are met.

### Infections or Predisposition to Infections:

8. Has a history of, or ongoing, chronic or recurrent infectious disease, including but not limited to, chronic renal infection, chronic chest infection (e.g., bronchiectasis), recurrent urinary tract infection (e.g., recurrent pyelonephritis or chronic nonremitting cystitis), or open, draining, or infected skin wounds or ulcers.
9. Has current signs or symptoms of a clinically significant infection. Established non-serious infections (e.g., acute upper respiratory tract infection, simple urinary tract infection) need not be considered exclusionary at the discretion of the investigator.
10. Has a history of serious infection (e.g., hepatitis, sepsis, pneumonia, or pyelonephritis), including any infection requiring hospitalization or IV antibiotics, for 8 weeks before baseline.
11. Has evidence of a herpes zoster infection within 8 weeks before baseline.
12. Has a history of latent or active granulomatous infection, including histoplasmosis or coccidioidomycosis, prior to screening. Participants with radiographic evidence of possible prior histoplasmosis or coccidioidomycosis will be excluded.
13. Has a chest radiograph within 12 weeks prior to the first administration of study intervention that shows an abnormality suggestive of a malignancy or current active infection, including TB.
14. Has or has had a nontuberculous mycobacterial infection or clinically significant opportunistic infection (e.g., cytomegalovirus colitis, pneumocystosis, invasive aspergillosis).
15. Participants must undergo screening for human immunodeficiency virus (HIV). Any participant who has a history of HIV antibody positivity, or tests positive for HIV at screening, is not eligible for this study.
16. Participants who are seropositive for antibodies to hepatitis C virus (HCV), unless they have 2 negative HCV RNA test results at least 6 months apart after completing antiviral treatment and prior to screening, and have a third negative HCV RNA test result at screening.
17. Tests positive for hepatitis B virus (HBV) infection.
Note: For participants who are not eligible for this study due to HIV, HCV, HBV, or TB test results, consultation with a physician with expertise in the treatment of those infections is recommended.
18. Has received, or is expected to receive, any live virus or bacterial vaccination within 12 weeks before the first administration of study intervention. For Bacille Calmette-Guerin (BCG) vaccine, see Exclusion Criterion 14.
19. Has had a BCG vaccination within 12 months of screening.

### Malignancy or Increased Potential for Malignancy

20. Currently has a malignancy or has a history of malignancy within 5 years before screening (with the exception of a nonmelanoma skin cancer that has been adequately treated with no evidence of recurrence for at least 3 months [defined as a minimum of 12 weeks] before the first study intervention administration or cervical carcinoma in situ that has been treated with no evidence of recurrence for at least 3 months before the first study intervention administration).
21. Has a known history of lymphoproliferative disease, including monoclonal gammopathy of unknown significance, lymphoma, or signs and symptoms suggestive of possible lymphoproliferative disease, such as lymphadenopathy, hepatomegaly, or splenomegaly, or monoclonal gammopathy of undetermined significance.

### Coexisting Medical Conditions or Past Medical History

22. Has a history of severe, progressive, or uncontrolled renal, genitourinary, hepatic, hematologic, endocrine, cardiac, vascular, pulmonary, rheumatologic, neurologic, psychiatric, or metabolic disturbances, or signs and symptoms thereof.
23. Has a transplanted organ (with exception of a corneal transplant >12 weeks before screening).
24. Is unable or unwilling to undergo multiple venipunctures because of poor tolerability or lack of adequate venous access.
25. Is known to have had a history of drug or alcohol abuse according to Diagnostic and Statistical Manual of Disorders (5th edition) (DSM-V) criteria within 12 months before baseline.
26. Has unstable suicidal ideation or suicidal behavior in the last 6 months that may be defined as a Columbia-Suicide Severity Rating Scale (C-SSRS) rating at screening of: Suicidal Ideation with Intention to Act (“Ideation level 4”), Suicidal Ideation with Specific Plan and Intent (“Ideation level 5”), or suicidal behavior (actual suicide attempt, interrupted suicide attempt, aborted suicide attempt, or preparatory behaviors for making a suicide attempt), and is considered to be at risk by the investigator based on an evaluation by a mental health professional. In addition, participants with C-SSRS ratings of Wish to be Dead (“Ideation level 1”), Non-Specific Active Suicidal Thoughts (“Ideation level 2”), Active Suicidal Ideation with Any Methods (Not Plan) without Intent to Act (“Ideation level 3”) or non-suicidal self-injurious behavior who are determined to be at risk by the investigator may not be randomized.
27. Has known allergies, hypersensitivity, or intolerance to guselkumab or ustekinumab or any of their excipients (see guselkumab IB and ustekinumab IB).
28. Is a woman who is pregnant, or breastfeeding, or planning to become pregnant while enrolled in this study or within 16 weeks after the last administration of study intervention.
29. Is a man who plans to father a child while enrolled in this study or within 16 weeks after the last administration of study intervention.

### General

30. Is currently enrolled in or intends to participate in any other study using an investigational agent or procedure during participation in this study.
31. Has any condition for which, in the opinion of the investigator, participation would not be in the best interest of the participant (eg, compromise the well-being) or that could prevent, limit, or confound the protocol-specified assessments.
32. Is an employee of the investigator or study site, with direct involvement in the proposed study or other studies under the direction of that investigator or study site, as well as family members of the employees or the investigator.
NOTE: Investigators should ensure that all study enrollment criteria have been met at screening. If a participant's clinical status changes (including any available laboratory results or receipt of additional medical records) after screening but before the first dose of study intervention is given such that he or she no longer meets all eligibility criteria, then the participant should be excluded from participation in the study.

### Study Interventions Administered

In both the Phase 2 and Phase 3 portions of the protocol:
All participants will receive 2 IV infusions at Week 0 (either active or placebo) and 1 IV infusion at Weeks 4, 8, and 12 (either active or placebo).
All participants will receive 1 SC injection (either active or placebo) at Week 8 and up to 3 SC injections (either active or placebo) at each visit from Week 12 to Week 140.

Intravenous study intervention should be administered over a period of not less than 1 hour, and not more than 2 hours. The infusion should be completed within 6 hours of preparation. Since multiple SC injections may be administered within the administration visit, each injection of study intervention should be given at a different location of the body.

### Concomitant Medications

Participants who are receiving oral 5-ASA compounds, oral corticosteroids, conventional immunomodulators (ie, AZA, 6-MP, or MTX), antibiotics, and/or enteral nutrition for the treatment of Crohn's disease at baseline should maintain a stable dose for the specified period before baseline, as defined in the Inclusion Criteria.

In general, participants who are receiving these medications for Crohn's disease at baseline (i.e., Week 0) of all 3 studies should maintain a stable dose through Week 48, with the exception of oral corticosteroids. Therapies can only be discontinued or reduced in dose after Week 0 if investigator judgment requires it because of toxicity or other medical necessity; even if the toxicity resolves, the therapy should not be restarted. Corticosteroids must be maintained at baseline doses through Week 12, and all participants must begin tapering corticosteroids at Week 12, unless medically not feasible.

### Week 0 Through Week 48

From Week 0 through Week 48 of each study, enrolled participants should not initiate any of the following concomitant Crohn's disease-specific medical therapies:
Oral or rectal 5-ASA compounds.
Immunomodulators (ie, AZA, 6-MP, or MTX).
Oral, parenteral, or rectal corticosteroids, including budesonide and beclomethasone dipropionate.
Antibiotics as a primary treatment for Crohn's disease.
Total parenteral nutrition or enteral nutrition as a treatment for Crohn's disease.

If the above medical therapies are initiated or medication doses are changed based on medical necessity as assessed by the investigator, participants should continue to attend all study visits and have all assessments. While this does not represent a deviation from the study protocol and the participants may remain on their assigned therapy (guselkumab, ustekinumab, or placebo), it may be considered a treatment failure. Treatment failures will be defined in the SAP.

### Week 12 and Through Week 48

From Week 12 through Week 48 of each study, participants may transiently use (i.e., for <4 weeks) increased doses of corticosteroids for reasons other than loss of response to treatment for Crohn's disease (eg, stress doses of corticosteroids for surgery, asthma, adrenocortical insufficiency).

### During Treatment Phase of LTE (Ie, Week 48 Through Week 144):

Concomitant therapies for Crohn's disease including 5-ASAs, corticosteroids, antibiotics, and immunomodulators (ie, AZA, 6-MP, or MTX), and/or total parental or enteral nutrition may be administered and changed at the discretion of the investigator.

### Oral Corticosteroids Tapering

At Week 12, all participants who were taking corticosteroids at Week 0 must begin tapering corticosteroids. This tapering is mandatory, unless not medically feasible, and should follow the recommended schedule shown in Table 6. If participants experience worsening of their disease activity while tapering corticosteroids, further dose decreases may be suspended, and/or their oral corticosteroid dose may be temporarily increased if deemed necessary by the investigator. The oral corticosteroid dose, however, may not be increased above the Week 0 dose unless due to medical necessity. For participants whose corticosteroid taper is interrupted, investigators are encouraged to resume tapering within 4 weeks. Tapering may exceed this schedule only if warranted by medical necessity (eg, participant experiencing corticosteroid-related side effects).

### Prohibited Concomitant Medications

Participants who initiate the following treatments during study participation will have their study intervention discontinued:
Immunomodulatory agents other than AZA, 6-MP, or MTX (including, but not limited to, 6-TG, cyclosporine, mycophenolate mofetil, tacrolimus, and sirolimus).
Immunomodulatory biologic agents (including, but not limited to, TNF antagonists, natalizumab, ustekinumab, rituximab, vedolizumab). Ustekinumab is permitted in this study only in participants randomly assigned to ustekinumab and only as stipulated in this protocol.
Experimental Crohn's disease medications (including, but not limited to, upadacitinib, filgotinib, ozanimod, etrolizumab, brazikumab, mirakizumab [formerly LY-3074828], risankizumab, GS-5745).
Thalidomide or related agents.

### Efficacy Assessments

Efficacy evaluations will include the following:
CDAI
PRO-2 (the unweighted CDAI components of the total number of liquid or very soft stools and the abdominal pain score)
Endoscopic assessments of the intestinal mucosa based on the presence and absence of mucosal ulcerations and the SES-CD, and histologic assessments based on the Global Histology Activity Score (GHAS)
Inflammatory PD markers including CRP and fecal calprotectin
Fistula assessment
Patient-reported outcome (PRO) measures to assess HRQOL outcomes (ie, IBDQ, PROMIS-29, and PROMIS Fatigue 7-item Short Form [7a], and EQ-5D-5L), and health economics outcomes (ie, WPAI-CD)
Exploratory patient-reported symptom measures including BSFS, AP-NRS, Patient's Global Impression of Severity (PGIS) of Crohn's Disease, and Patient's Global Impression of Change (PGIC) of Severity of Crohn's Disease

The CDAI be assessed by collecting information on 8 different Crohn's disease-related variables: extra-intestinal manifestations, abdominal mass, weight, hematocrit, total number of liquid or very soft stools, abdominal pain/cramping, use of antidiarrheal drug(s) and/or opiates, and general well-being. The last 4 variables are scored over 7 days by the participant on a diary card that participants are to complete on a daily basis. The PRO-2 includes the unweighted CDAI components of the total number of liquid or very soft stools and the AP score.

Endoscopic assessments of the intestinal mucosa will be evaluated during ileocolonoscopy in all participants. A video ileocolonoscopic examination will be performed at Screening, Week 12, Week 48, and Week 96. An optional sub-study involving a Week 4 evaluation will be performed in consenting participants in addition to the above specified evaluations. Video endoscopies will be assessed by a central facility that will be blinded to treatment group and visit. A complete video endoscopic examination does not require assessment of the terminal ileum if it cannot be visualized. The SES-CD score will be used to evaluate Endoscopic Improvement. The SES-CD is based on the evaluation of 4 endoscopic components (presence/size of ulcers, proportion of mucosal surface covered by ulcers, proportion of mucosal surface affected by any other lesions, and presence/type of narrowing/strictures) across 5 ileocolonic segments. Each endoscopic component is scored from 0 to 3 for each segment, resulting in a total score of up to 15 for each component, except for the narrowing component which can only attain a maximum total score of 11 because by definition, the presence of a narrowing that cannot be passed can be only observed once. In summary, an overall total SES-CD score is derived from the sum of all the component scores and can range from 0 to 56). Endoscopic healing, which is traditionally defined as the resolution (absence) of mucosal ulcers in response to a therapeutic intervention, will also be assessed.

Histologic assessments will be performed using biopsy samples collected during ileocolonoscopy. Biopsy samples will be collected at screening, Week 12, Week 48, and Week 96 from each of 3 predefined anatomic locations: the terminal ileum, splenic flexure, and rectum. An optional sub-study involving a Week 4 evaluation will be performed in consenting participants in addition to the above-specified evaluations. The biopsy samples collected post-baseline will be obtained near where the screening biopsy samples were collected from each of the 3 predefined locations. Histologic assessments will be conducted by a central reader who is blinded to treatment groups and visit. The Global Histology Activity Score (GHAS) will be used to evaluate histologic improvements and healing.5 Analyses will be specified in the SAP.

Fistula assessment will be performed in all participants on an ongoing basis throughout the duration of the studies. All participants will be assessed for fistulas at baseline. For participants with fistulizing disease, fistula closure will be assessed during the studies. Enterocutaneous fistulas (eg, perianal and abdominal) will be considered no longer draining (ie, closed) when there is absence of drainage despite gentle compression. Rectovaginal fistulas will be considered closed based on either physical examination or absence of relevant symptoms (eg, passage of rectal material or flatus from the vagina).

Patient-reported outcome measures will be evaluated at visits as indicated in the Schedule of Activities (Section 1.3):
The IBDQ is a validated, 32-item, self-reported questionnaire for participants with IBD to evaluate PROs across 4 dimensions: bowel symptoms (loose stools, abdominal pain), systemic symptoms (fatigue, altered sleep pattern), social function (work attendance, need to cancel social events), and emotional function (anger, depression, irritability).11 Scores range from 32 to 224, with higher scores indicating better outcomes.
The PROMIS-29 is a validated general health profile instrument that is not disease-specific. It is a collection of short forms containing 4 items for each of 7 domains (depression, anxiety, physical function, pain interference, fatigue, sleep disturbance, and ability to participate in social roles and activities). PROMIS-29 also includes an overall average pain intensity 0-10 numeric rating scale (NRS).
The PROMIS Fatigue 7-items Short Form (PROMIS Fatigue Short Form 7a) contains 7 items evaluating fatigue-related symptoms (ie, tiredness, exhaustion, mental tiredness, and lack of energy) and associated impacts on daily activities (ie, activity limitations related to work, self-care, and exercise). PROMIS Fatigue Short Form 7a has a recall period of past 7 days. Compared to the fatigue scale of PROMIS-29, PROMIS Fatigue Short Form 7a provides additional information to evaluate severity of fatigue.
The EQ-5D-5L is a validated instrument consisting of the EuroQol five dimensions descriptive system (EQ-5D) and the EuroQol visual analog scale (EQ-VAS). The descriptive system comprises 5 dimensions (mobility, self-care, usual activities, pain/discomfort, anxiety/depression). Each dimension has 5 levels: no problems, slight problems, moderate problems, severe problems, and extreme problems. The respondent is asked to indicate his/her health state by checking the most appropriate statement in each of the 5 dimensions. The EQ-VAS records the respondent's self-rated health on a 20-cm vertical, visual analog scale with endpoints labeled ‘the best health you can imagine’ and ‘the worst health you can imagine’. The respondents mark an “X” on the scale to indicate their health TODAY and then write the number marked on the scale in the box.
The WPAI-CD is a validated instrument created as a patient-reported quantitative assessment of the amount of absenteeism, presenteeism, and daily activity impairment attributable to Crohn's disease. The WPAI-CD consists of 6 questions to determine employment status, hours missed from work due to Crohn's disease, hours missed from work for other reasons, hours worked, the degree to which Crohn's disease affected work productivity while at work, and the degree to which Crohn's disease affected activities outside of work. Four scores are derived: percentage of absenteeism, percentage of presenteeism (reduced productivity while at work), an overall work impairment score that combines absenteeism and presenteeism, and percentage of impairment in activities performed outside of work. Higher scores indicate greater impairment.

Exploratory patient-reported symptom measures will be evaluated at visits as indicated in the Schedule of Activities:
The BSFS is a medical aid to classify the form (or consistency) of human feces into 7 categories.14 It has been used as a research tool to evaluate the effectiveness of treatments for various diseases of the bowel (eg, irritable bowel syndrome [IBS]). Participants will complete the BSFS as a daily diary entry from Week 0 through Week 48.
The AP-NRS is an 11-point (0-10) scale that will be used to evaluate abdominal pain. The score of 0 represents “no abdominal pain” and the score of 10 represents the “worst possible abdominal pain” with greater scores indicating greater pain severity and intensity. Participants will complete the AP-NRS as a daily diary entry from Week 0 through Week 48, selecting only one number that best reflects their pain at its worst.
PGIS of Crohn's Disease: Participants will rate their Crohn's disease activity at baseline and each visit using a 5-point scale (“None”, “Mild”, “Moderate”, “Severe” and “Very Severe”). The PGIS will be used as an anchor to establish and or validate response criteria of other clinical endpoints.
PGIC of Severity of Crohn's Disease: Participants' perceived change (improvement or deterioration) in the severity of their Crohn's disease will be assessed using the PGIC. Participants will rate how their Crohn's disease has changed since the beginning of the study using a 7-point scale ranging from “a lot better now” to “a lot worse now” with a neutral center point (“neither better nor worse”). The PGIC will be used as an anchor to establish and or validate response criteria of other clinical endpoints.

### Safety Assessments

Adverse events will be reported and followed by the investigator. Any clinically relevant changes occurring during the study must be recorded in the Adverse Event section of the eCRF. Any clinically significant abnormalities persisting at the end of the study/early withdrawal will be followed by the investigator until resolution or until a clinically stable endpoint is reached.

The study will include the following evaluations of safety and tolerability according to the time points specified:

### Electrocardiogram

A 12-lead electrocardiogram (ECG) will be performed at screening.

During the collection of ECGs, participants should be in a quiet setting without distractions (eg, television, cell phones). Participants should rest in a supine position for at least 5 minutes before ECG collection and should refrain from talking or moving arms or legs. If blood sampling or vital sign measurement is scheduled for the same time point as ECG recording, the procedures should be performed in the following order: ECG(s), vital signs, blood draw.

### Physical Examination

Physical examinations will be performed as specified in the Schedule of Activities. While assessment of the participants for safety and efficacy requires some physical examination by an investigator at all visits, a more complete, detailed physical exam will be performed at specified visits.

### Height and Weight

Height and weight will be measured as specified in the Schedule of Activities. Subjects will be instructed to remove shoes and outdoor apparel and gear prior to these measurements.

### Vital Signs

Vital signs (including temperature, pulse/heart rate, respiratory rate, and blood pressure) will be obtained before and approximately every 30 minutes during every IV infusion, and at approximately 30-minute intervals after completion of the final IV infusion. Vital signs should be obtained before and approximately 30 minutes after the final SC injection.

### Infections

Study intervention administration should not be given to a participant with a clinically important, active infection. Investigators are required to evaluate participants for any signs or symptoms of infection at scheduled visits (see Schedule of Activities, Section 1.3). If a participant develops a serious infection, including but not limited to sepsis or pneumonia, discontinuation of study treatment (ie, no further study intervention administrations) must be considered.

### Tuberculosis Evaluation(s)

### Initial Tuberculosis Evaluation

Participants must undergo testing for TB and their medical history assessment must include specific questions about a history of TB or known occupational or other personal exposure to individuals with active TB. The participant should be asked about past testing for TB, including chest radiograph results and responses to tuberculin skin or other TB testing. Investigators have the option to use both the QuantiFERON-TB Gold test and the tuberculin skin test to screen for latent TB if they believe, based on their judgment, that the use of both tests is clinically indicated in order to evaluate a participant who has high risk of having latent TB. If either the QuantiFERON-TB Gold test or the tuberculin skin test is positive, the participant is considered to have latent TB infection for the purposes of eligibility for this study.

Participants with a negative QuantiFERON-TB Gold test result (and a negative tuberculin skin test result in countries in which the QuantiFERON-TB Gold test is not approved/registered or the tuberculin skin is mandated by local health authorities) are eligible to continue with pre-randomization procedures. Participants with a newly identified positive QuantiFERON-TB Gold (or tuberculin skin) test result must undergo an evaluation to rule out active TB and initiate appropriate treatment for latent TB. Appropriate treatment for latent TB is defined according to local country guidelines for immunocompromised patients. If no local country guidelines for immunocompromised patients exist, US guidelines must be followed, or the participant will be excluded from the study.

A participant whose first QuantiFERON-TB Gold test result is indeterminate should have the test repeated. In the event that the second QuantiFERON-TB Gold test result is also indeterminate, the participant may be enrolled without treatment for latent TB if active TB is ruled out, their chest radiograph shows no abnormality suggestive of TB (active or old, inactive TB) and the participant has no additional risk factors for TB as determined by the investigator. This determination must be promptly reported to the sponsor's or designee's medical monitor and recorded in the participant's source documents and initialed by the investigator.

### Tuberculosis Evaluation

### Early Detection of Active Tuberculosis

To aid in the early detection of TB reactivation or new TB infection during study participation, participants must be evaluated for signs and symptoms of active TB at scheduled visits or by telephone contact approximately every 8 to 12 weeks. The following series of questions is suggested for use during the evaluation:
“Have you had a new cough of >14 days' duration or a change in a chronic cough?”
“Have you had any of the following symptoms:
   Persistent fever?
   Unintentional weight loss?
   Night sweats?”
“Have you had close contact with an individual with active TB?” (If there is uncertainty as to whether a contact should be considered “close,” a physician specializing in TB should be consulted.)

If the evaluation raises suspicion that a participant may have TB reactivation or new TB infection, an immediate and thorough investigation should be undertaken, including, where possible, consultation with a physician specializing in TB. Investigators should be aware that TB reactivation in immunocompromised participants may present as disseminated disease or with extrapulmonary features. Participants with evidence of active TB should be referred for appropriate treatment. Participants who experience close contact with an individual with active TB during the conduct of the study must have a repeat chest radiograph, a repeat QuantiFERON TB Gold test, a repeat tuberculin skin test in countries in which the QuantiFERON-TB Gold test is not approved/registered or the tuberculin skin test is mandated by local health authorities, and, if possible, referral to a physician specializing in TB to determine the participant's risk of developing active TB and whether treatment for latent TB is warranted.

Study intervention administration should be interrupted during the investigation. A positive QuantiFERON-TB Gold test or tuberculin skin test result should be considered detection of latent TB. If the QuantiFERON-TB Gold test result is indeterminate, the test should be repeated as outlined in Appendix 5 (Section 10.5). Participants should be encouraged to return for all subsequent scheduled study visits according to the protocol. Subjects who discontinue treatment for latent TB prematurely or who are noncompliant with therapy must immediately discontinue further administration of study intervention and be encouraged to return for all subsequent scheduled study visits according to the Schedule of Activities (Section 1.3).

### Allergic Reaction

Before any SC injection or IV infusion, appropriately trained personnel and medications must be available to treat allergic reactions, including anaphylaxis. All participants must be observed carefully for symptoms of an allergic reaction (eg, urticaria, itching, hives). If a mild or moderate allergic reaction is observed, acetaminophen, nonsteroidal anti-inflammatory drugs, and/or diphenhydramine may be administered.

In the case of a severe allergic reaction (eg, anaphylaxis), SC aqueous epinephrine, corticosteroids, respiratory assistance, and other proper resuscitative measures are essential and must be available at the study site where the injections or infusions are being administered.

Participants who experience serious adverse reactions related to an injection or infusion should be discontinued from further study intervention administrations.

Participants who experience reactions following an injection or infusion that result in bronchospasm with wheezing and/or dyspnea that requires ventilatory support, or symptomatic hypotension with a decrease in systolic blood pressure greater than 40 mm Hg will not be permitted to receive additional study intervention.

Participants who experience reactions suggestive of serum sickness-like reactions (resulting in symptoms such as myalgia and/or arthralgia with fever and/or rash that are not representative of signs and symptoms of other recognized clinical syndromes) occurring 1 to 14 days after an injection of study intervention, should be discontinued from further study intervention administrations. Note that these symptoms may be accompanied by other events including pruritus, facial, hand, or lip edema, dysphagia, urticaria, sore throat, and/or headache.

### Adverse Events Temporally Related to Infusion

Any AE (except laboratory abnormalities) that occurs during or within 1 hour after the IV infusion of study intervention will be carefully evaluated. Minor infusion-related AEs may be managed by slowing the rate of the IV infusion and/or treating with antihistamines and/or acetaminophen (paracetamol) as clinically indicated. If an IV infusion of study intervention is stopped because of an AE that, in the opinion of the investigator, is not severe or does not result in a serious adverse event (SAE), the infusion may be restarted with caution.

### Injection-Site Reaction

An injection-site reaction is any adverse reaction at a SC study intervention injection site. Injection sites will be evaluated for reactions and any injection-site reaction will be recorded as an AE.

### Columbia-Suicide Severity Rating Scale (C—SSRS)

The C—SSRS defines 5 subtypes of suicidal ideation and 4 possible suicidal behaviors, as well as non-suicidal self-injurious behavior and completed suicide. It will be used as a screening tool to prospectively evaluate suicidal ideation and behavior in this study, as part of a comprehensive evaluation of safety. The C—SSRS is an investigator-administered questionnaire. Two versions of it will be used in this study: the ‘Baseline/Screening’ version of the C—SSRS will be conducted during the screening visit and the ‘Since Last Visit’ version of the C—SSRS will be completed at all other visits through the end of the study.

The investigator or trained study-site personnel will interview the participant and complete the C—SSRS. The C—SSRS will be provided in the local languages in accordance with local guidelines.

At screening, the C—SSRS will be the first assessment performed, before any other study procedure. At all subsequent visits, the C—SSRS will be performed according to the assessment schedule and should be performed after other PROs but before any other study procedure. Participants will be interviewed by the investigator or trained study-site personnel in a private, quiet place.

At the conclusion of each assessment, the trained personnel administering the C—SSRS will determine the level of suicidal ideation or behavior, if any. They will then determine the next course of action if any level of suicidal ideation or behavior is reported. The participant should not be released from the site until the C—SSRS has been reviewed by the investigator and the participant's risk has been assessed and follow-up determined, as appropriate.

At screening (within the last 6 months) and Week 0, participants with a C—SSRS rating of Suicidal Ideation with Intention to Act (“Ideation level 4”), Suicidal Ideation with Specific Plan and Intent (“Ideation level 5”), or suicidal behavior (actual suicide attempt, interrupted suicide attempt, aborted suicide attempt, or preparatory behaviors for making a suicide attempt), must be determined to not be at risk by the investigator based on an evaluation by a mental health professional (eg, psychiatrist, psychologist, or appropriately trained social worker or nurse) in order to be randomized.

Participants with C—SSRS ratings of Wish to be Dead (“Ideation level 1”), Non-Specific Active Suicidal Thoughts (“Ideation level 2”), Active Suicidal Ideation with Any Methods (Not Plan) without Intent to Act (“Ideation level 3”) or non-suicidal self-injurious behavior must be determined not to be at risk by the investigator in order to be randomized. Any questions regarding eligibility of such participants should be discussed with the medical monitor or designee.

For each assessment after Week 0, the following actions should be taken, if applicable:
No suicidal ideation or behaviors (including self-injurious behavior without suicidal intent): No further action is needed.
Suicidal ideation levels 1-3 or non-suicidal self-injurious behavior: Participant risk is assessed by the investigator.
Suicidal ideation levels 4 or 5 or any suicidal behavior: Participant risk assessed and referral to a mental health professional.

Interruption or the discontinuation of study treatment should be considered for any participant who reports Suicidal Ideation with Intention to Act (“Ideation level 4”), Suicidal Ideation with Specific Plan and Intent (“Ideation level 5”), or suicidal behavior (actual suicide attempt, interrupted suicide attempt, aborted suicide attempt, or preparatory behaviors for making a suicide attempt) on a post-baseline C-SSRS assessment and who is deemed to be at risk by the investigator based upon evaluation by a mental health professional. If a participant can be adequately treated with psychotherapy and/or pharmacotherapy then the participant, at the discretion of the investigator, may be continued with treatment if agreed to by the medical monitor or designee. Discussion of such participants with the medical monitor or designee is required.

Any C—SSRS finding, which in the opinion of the investigator is new or considered to be a worsening and clinically significant, should be reported on the AE eCRF, Adverse Events: Definitions and Procedures for Recording, Evaluating, Follow-up, and Reporting).

### Clinical Safety Laboratory Assessments

Blood samples for serum chemistry and hematology will be collected. The investigator must review the laboratory results, document this review, and record any clinically relevant changes occurring during the study in the AE section of the eCRF. The laboratory reports must be filed with the source documents.

The following tests will be performed by the central laboratory unless otherwise specified or approved by the medical monitor.
Hematology assessments will include but are not limited to the following: hemoglobin, hematocrit, platelet count, total and differential WBC count.
Blood chemistry assessments will include but are not limited to the following: chemistry panel (total and direct bilirubin, ALT, AST, alkaline phosphatase, albumin, total protein, calcium, phosphate, sodium, potassium, chloride, blood urea nitrogen/urea, and creatinine).

A medical monitor or delegate and the clinical site will be notified if pre-specified abnormal laboratory values defined in the Laboratory Manual are identified in any participant during the conduct of the study.
Serology: HIV antibody, HBV antibodies and surface antigen, and HCV antibody
Abnormal liver function tests: If laboratory testing for a subject who is enrolled in the study and receiving study intervention reveals an increase of serum aminotransferases (ALT or AST) to >3×ULN and an increase of bilirubin to >2×ULN, study agent should be suspended immediately. In addition, laboratory tests for ALT, AST, alkaline phosphatase, and total bilirubin should be confirmed by a retest within 24 hours if possible, but no later than 72 hours following notification of test results.
Pregnancy testing: Female participants of childbearing potential will undergo a urine pregnancy test at screening before each study intervention administration, at a SID visit, and at the FES visit.

### Immunogenicity Assessments (Antibodies to Guselkumab and Ustekinumab)

Serum samples will be screened for antibodies binding to guselkumab or ustekinumab and the titer of confirmed positive samples will be reported as applicable. Other analyses may be performed to further characterize the immunogenicity of guselkumab or ustekinumab. Antibodies to guselkumab or ustekinumab will be evaluated on blood drawn from all participants. Additionally, samples should also be collected at the final visit for participants who terminate from the study. These samples will be tested by the sponsor or sponsor's designee. Genetic analyses will not be performed on these serum samples. Participant confidentiality will be maintained.

### Evaluations

At visits where antibodies to study intervention will be evaluated in addition to serum concentration of study intervention, 1 venous blood sample of sufficient volume should be collected. Each serum sample will be divided into 3 aliquots (1 each for serum concentration of study intervention, antibodies to study intervention, and a back-up).

### Analytical Procedures

The detection and characterization of antibodies to guselkumab and ustekinumab will be performed using validated assay methods by or under the supervision of the sponsor.

### Medication Review

Concomitant medications will be reviewed at each visit.

### Adverse Events and Serious Adverse Events

Timely, accurate, and complete reporting and analysis of safety information from clinical studies are crucial for the protection of participants, investigators, and the sponsor, and are mandated by regulatory agencies worldwide. The sponsor has established Standard Operating Procedures in conformity with regulatory requirements worldwide to ensure appropriate reporting of safety information; all clinical studies conducted by the sponsor or its affiliates will be conducted in accordance with those procedures.

Adverse events will be reported by the participant (or, when appropriate, by a caregiver, surrogate, or the participant's legally acceptable representative) for the duration of the study. Anticipated events will be recorded and reported.

### Time Period and Frequency for Collecting Adverse Event and Serious Adverse Event Information

### All Adverse Events

All AEs and special reporting situations, whether serious or non-serious, will be reported from the time a signed and dated ICF is obtained until completion of the participant's last study-related procedure, which may include contact for follow-up of safety. Serious adverse events, including those spontaneously reported to the investigator within 16 weeks after the last dose of study intervention, must be reported using the Serious Adverse Event Form. The sponsor will evaluate any safety information that is spontaneously reported by an investigator beyond the time frame specified in the protocol.

### Serious Adverse Events

All SAEs occurring during the study must be reported to the appropriate sponsor or designee contact person by study-site personnel within 24 hours of their knowledge of the event. Information regarding SAEs will be transmitted to the sponsor or designee using the Serious Adverse Event Form, which must be completed and reviewed by a physician from the study site, and transmitted to the sponsor or designee within 24 hours.

### Follow-Up of Adverse Events and Serious Adverse Events

Adverse events, including pregnancy, will be followed by the investigator.

### Regulatory Reporting Requirements for Serious Adverse Events

The sponsor assumes responsibility for appropriate reporting of AEs to the regulatory authorities. The sponsor will also report to the investigator (and the head of the investigational institute where required) all SUSARs. The investigator (or sponsor where required) must report SUSARs to the appropriate IEC/IRB that approved the protocol unless otherwise required and documented by the IEC/IRB. A SUSAR will be reported to regulatory authorities unblinded. Participating investigators and IEC/IRB will receive a blinded SUSAR summary, unless otherwise specified.

### Pregnancy

All initial reports of pregnancy in female participants or partners of male participants must be reported to the sponsor or designee by the study-site personnel within 24 hours of their knowledge of the event using the appropriate pregnancy notification form. Abnormal pregnancy outcomes (eg, spontaneous abortion, fetal death, stillbirth, congenital anomalies, ectopic pregnancy) are considered SAEs and must be reported using the Serious Adverse Event Form. Any participant who becomes pregnant during the study must discontinue further study intervention.

Follow-up information regarding the outcome of the pregnancy and any postnatal sequelae in the infant will be required.

### Events of Special Interest

Any newly identified malignancy or case of active TB occurring after the first study intervention administration(s) in participants participating in this clinical study must be reported by the investigator. Investigators are also advised that active TB is considered a reportable disease in most countries. These events are to be considered serious only if they meet the definition of an SAE.

### Treatment of Overdose

For this study, any dose of study intervention greater than the highest dose at a single dosing visit specified in this protocol will be considered an overdose. The sponsor does not recommend specific intervention for an overdose.

In the event of an overdose, the investigator or treating physician should:
Contact the Medical Monitor immediately.
Closely monitor the participant for AE/SAE and laboratory abnormalities.
Document the quantity of the excess dose in the eCRF.

Decisions regarding dose interruptions or modifications will be made by the investigator in consultation with the Medical Monitor based on the clinical evaluation of the participant.

### Pharmacokinetics

Serum samples will be used to evaluate the PK of guselkumab and ustekinumab. Samples collected for the analyses of serum concentrations of guselkumab and ustekinumab may additionally be used to evaluate safety or efficacy aspects that address concerns arising during or after the study period, or for the evaluation of relevant biomarkers. Genetic analyses will not be performed on these serum samples. Participant confidentiality will be maintained.

### Evaluations

At visits where only serum concentration of study intervention will be evaluated (ie, no antibodies to study intervention will be evaluated), 1 venous blood sample of sufficient volume should be collected, and each serum sample should be divided into 2 aliquots (1 for serum concentration of study intervention, and a back-up). At visits where serum concentration of study intervention and antibodies to study intervention will be evaluated, 1 venous blood sample of sufficient volume should be collected. Each serum sample will be divided into 3 aliquots (1 each for serum concentration of study intervention, antibodies to study intervention, and a back-up).

### Analytical Procedures

Serum samples will be analyzed to determine concentrations of guselkumab and ustekinumab using respective validated, specific, and sensitive methods by or under the supervision of the sponsor's respective assay methods.

### Pharmacokinetic Parameters

Serum samples will be used to evaluate various guselkumab PK parameters based on blood drawn from all participants according to the Schedule of Activities.

### Pharmacodynamics

Inflammatory PD markers will be evaluated using blood samples collected at visits Post-baseline PD test results will not be released to the investigators by the central laboratory.
CRP has been demonstrated to be useful as a marker of inflammation in patients with IBD. In Crohn's disease, elevated CRP concentrations have been associated with severe clinical activity, elevated sedimentation rate, and active disease as detected by colonoscopy. Blood samples for the measurement of CRP will be collected from all participants. CRP will be evaluated using a validated, high-sensitivity assay.
Fecal calprotectin has been demonstrated to be a sensitive and specific marker in identifying intestinal inflammation and response to treatment in patients with IBD.3 Stool samples for fecal calprotectin concentration will be collected from all participants. The assay for fecal calprotectin concentration will be performed using a validated method. Additional tests may also be performed on the stool samples for additional markers related to intestinal inflammation and treatment response such as the microbiome.

### Genetics

A pharmacogenomic blood sample will be collected from participants who consent separately to this component of the study to allow for pharmacogenomic research, as necessary where local regulations permit. Participation in pharmacogenomic research is optional.

Genetic (DNA) variation may be an important contributory factor to interindividual variability in drug response and associated clinical outcomes. Genetic factors may also serve as markers for disease susceptibility and prognosis, and may identify population subgroups that respond differently to an intervention.

DNA samples will be analyzed for identification of genetic factors that may be associated with clinical response. This research may consist of the analysis of 1 or more candidate genes, assessment of Single Nucleic Polymorphisms (SNPs), or analysis of the entire genome (as appropriate) in relation to guselkumab or ustekinumab intervention and/or Crohn's disease. Whole blood samples of approximately 10 mL will be collected for genetic analyses.

### Phase 2 Dose-Ranging Study (GALAXI 1)

The primary hypothesis is that guselkumab is superior to placebo as assessed by the reduction from baseline in CDAI at Week 12.

### Phase 3 Dose-Confirming Studies (GALAXI 2 and GALAXI 3)

The primary hypothesis is that guselkumab treatment is superior to placebo as assessed by the proportion of participants achieving clinical remission at Week 12.

For the major secondary hypotheses for comparison with ustekinumab, while the ultimate goal is to demonstrate that the efficacy of guselkumab is superior to ustekinumab, an initial test for non-inferiority is included because the overall profile of guselkumab may be favorable compared with ustekinumab (in terms of overall efficacy and safety), even if final results only indicate the relative efficacy Sample Size Determination

### Assumptions

Data from several sources informed the underlying assumptions for sample size determination in Phase 2 and Phase 3, as summarized in the below sections. These include the ustekinumab Crohn's disease Phase 3 program consisting of 3 studies (ie, CNTO1275CRD3001, CNTO1275CRD3002, and CNTO1275CRD3003), a program conducted by the sponsor in participants with Crohn's disease who had previously failed or were intolerant to TNF-antagonist therapy (referred to as TNF-Failure herein) or had previously failed or were intolerant to conventional therapies (referred to as CON-Failure herein), and the data from a risankizumab Crohn's disease Phase 2 study in which the majority of participants were those who had previously failed or were intolerant to biologic therapies (referred to as BIO-Failure herein).

### Clinical Remission at Week 12

Assumptions for the BIO-Failure population at Week 12 were based on the following:
In CNTO1275CRD3001, the proportions of participants in clinical remission (CDAI<150) at Week 8 were 7.3% and 20.9% for placebo and ustekinumab ˜6 mg/kg, respectively, for a treatment difference of 13.6%.8
Based on a clinical remission rate of 15% for placebo at Week 12, the risankizumab Phase 2 study suggested an approximate 9% difference in clinical remission between 200 mg IV and placebo, and an approximate 21% difference between 600 mg IV and placebo at Week 12.7 Based on these data, the clinical remission rates are assumed to be 10% for placebo, 20% for guselkumab 200 mg IV, and 30% for guselkumab 600 mg IV at Week 12 in the BIO-Failure population.

Assumptions for the CON-Failure population at Week 12 were based on the following:
In CNTO1275CRD3002, the proportions of participants in clinical remission at Week 8 were 19.6% and 40.2% for placebo and ustekinumab ˜6 mg/kg, respectively, for a treatment difference of 20.6%.8
No data are currently available for guselkumab or other anti-IL-23 agents in the CON-Failure population. Based on the data from CNTO1275CRD3002 and historical biologic studies in similar populations, it is reasonable to assume a greater treatment effect difference between active and placebo in the CON-Failure population compared with that observed in a BIO-failure population. In addition, the dose-response trend in the CON-Failure population is assumed to be similar to that observed in the BIO-Failure population.

Based on these data and assumptions, the clinical remission rates are assumed to be 20% for placebo, 40% for guselkumab 200 mg IV, and 50% for guselkumab 600 mg IV in the CON-Failure population.

In the absence of data for the 1200 mg IV dose from guselkumab or from other anti-IL-23 agents, to be conservative, the clinical remission rate for guselkumab 1200 mg IV is assumed to be similar to that for guselkumab 600 mg IV, at a minimum, for both BIO-Failure and CON-Failure populations.

Taking into account a mixed BIO-Failure/CON-Failure population, assumptions for the overall randomized population at Week 12 were based on the following:
Based on the ratio of a minimum of 25% and up to 50% of participants in the CON-Failure patient population, the proportions of participants in clinical remission at Week 12 is expected to be approximately 12% to 15% for placebo, approximately 25% to 30% for guselkumab 200 mg IV, and approximately 35% to 40% for both guselkumab 600 mg IV and guselkumab 1200 mg IV.

### Change in CDAI at Week 12

Assumptions for the BIO-Failure population and the CON-Failure population were based on the following:
In CNTO1275CRD3001, the mean CDAI change from baseline at Week 8 was −25.1 (SD=91.41) and −78.7 (SD=91.79) for the placebo and ustekinumab 6 mg/kg groups, respectively.8
In CNTO1275CRD3002, the mean CDAI change from baseline at Week 8 was −66.3 (SD=97.81) and −116.3 (SD=102.88) for the placebo and ustekinumab 6 mg/kg groups, respectively.8

Taking into account a mixed BIO-Failure/CON-Failure population, the mean CDAI reduction from baseline at Week 12 is expected to be approximately 45 to 50 for placebo, approximately 85 to 95 for guselkumab 200 mg IV, and approximately 105 to 115 for guselkumab 600 mg IV and guselkumab 1200 mg IV at Week 12 with a common SD of 100 (considering increased variability in a relatively smaller Phase 2 study).

### Clinical Remission at Week 48

Rates for clinical remission at Week 48 were derived by combining the randomized and non-randomized population in CNTO1275CRD3003, resulting in a clinical remission rate of 23% in TNF-Failure participants and 50% in CON-Failure participants for ustekinumab. As such, the overall randomized population with a minimum of 25% and up to 50% of the participants being from the CON-Failure population is expected to achieve approximately 30% to 36% clinical remission at Week 48 for ustekinumab. A meaningful difference of 15% in clinical remission between guselkumab and ustekinumab is assumed at Week 48.

### Power and Sample Size Calculations

### Phase 2 Dose-Ranging Study (GALAXI 1)

Power for Phase 2 was evaluated for the 2 analysis populations described below, using a 2-sample t-test (at the 0.05 level of significance) to detect a significant difference in the change from baseline in the CDAI score at Week 12 between the guselkumab high IV induction dose and placebo.

Assuming the mean CDAI reductions from baseline at Week 12 of approximately 105 to 115 in the guselkumab high IV induction dose group versus approximately 45 to 50 in the placebo group with a common SD of 100:
For the Initial Dose Decision Cohort: 50 participants in the guselkumab high IV induction dose group and 50 participants in the placebo group will provide greater than 80% power to detect a treatment difference between guselkumab and placebo at a Type 1 error rate controlled at α=0.05 (2-sided) (Table 8). With 5 dose groups, the total sample size for the Initial Dose Decision Cohort is 250 subjects.
For the Total Phase 2 Population: It is anticipated that 100 to 250 participants will be enrolled into the Transition Cohort by the time a dose decision is made for Phase 3. Thus, the sample size for the total Phase 2 study is expected to range from a minimum of 350 participants (70 per dose group) up to a maximum of 500 participants (100 per dose group). The power, based on the minimum number of participants, is greater than 90% for the change from baseline in the CDAI score at Week 12 and greater than 85% for clinical remission at Week 12 (Table 8). Table 8:

### Power to Detect a Treatment Effect of Guselkumab Versus Placebo Based on Mean Change in CDAI and Proportion of Participants Achieving Clinical Remission at Week 12

### Safety Analyses

### Adverse Events

The verbatim terms used in the eCRF by investigators to identify AEs will be coded using the Medical Dictionary for Regulatory Activities. Treatment-emergent AEs are AEs with onset during the intervention phase or that are a consequence of a pre-existing condition that has worsened since baseline. All reported treatment-emergent AEs will be included in the analysis. For each AE, the percentage of participants who experience at least 1 occurrence of the given event will be summarized by intervention group.

The following analyses of AEs will be used to assess the safety of participants:
Frequency and type of AEs.
Frequency and type of SAEs.
Frequency and type of reasonably related AEs as assessed by the investigator.
Frequency and type of AEs leading to discontinuation of study intervention.
Frequency and type of infections.

Frequency and type of AEs temporally associated with infusion.
Frequency and type of injection-site reactions.

Summaries, listings, datasets, or participant narratives may be provided, as appropriate, for those participants who die, who discontinue intervention due to an AE, or who experience a severe or a serious AE.

### Clinical Laboratory Tests

The following summaries of clinical laboratory tests will be used to assess participant safety:
Laboratory parameters and change from baseline in laboratory parameters (hematology and chemistry).
Summary of maximum NCI-CTCAE toxicity grade for post-baseline laboratory values (hematology and chemistry).

Listings of participants with any abnormal post-baseline laboratory values of NCI-CTCAE grade ≥2 will also be provided.

### Suicidal Ideation and Behavior

Suicidal ideation and behavior based on the C—SSRS and AEs will be summarized descriptively.

### Other Analyses

### Pharmacokinetic Analyses

Descriptive statistics of the serum guselkumab and ustekinumab concentrations will be calculated at each sampling time point. These concentrations will be summarized over time for each treatment group.

All concentrations below the lowest quantifiable concentration or missing data will be labeled as such in the concentration database or data presentations. Concentrations below the lowest quantifiable concentration will be treated as zero in the summary statistics.

A population PK analysis approach using nonlinear mixed-effects modeling will be used to evaluate guselkumab PK parameters. The influence of important covariates on the population PK parameter estimates may be evaluated. Details will be provided in a population PK analysis plan and the results of the population PK analysis will be presented in a separate technical report. Participants will be excluded from the PK analysis if their data do not allow for accurate assessment of the PK (eg, incomplete administration of the study intervention; missing time of study intervention administration). Detailed rules for the analysis will be specified in the SAPs.

### Immunogenicity Analyses

The incidence and titers of antibodies to guselkumab and ustekinumab will be summarized respectively for all participants who receive a dose of guselkumab or ustekinumab and have appropriate samples for detection of antibodies to guselkumab or ustekinumab (ie, participants with at least 1 sample obtained after their first dose of guselkumab or ustekinumab).

A listing of participants who are positive for antibodies to guselkumab or ustekinumab will be provided. The maximum titers of antibodies to guselkumab or ustekinumab will be provided for participants who are positive for antibodies to guselkumab or ustekinumab.

The incidence of neutralizing antibodies (NAbs) to guselkumab or ustekinumab will be summarized for participants who are positive for antibodies to guselkumab or ustekinumab and have samples evaluable for NAbs to guselkumab or ustekinumab.

### Biomarkers Analyses

Planned biomarker analyses may be deferred if emerging study data show no likelihood of providing useful scientific information. Any biomarker samples received by the contract vendor or sponsor after the cutoff date will not be analyzed, and therefore, excluded from the biomarker analysis.

Changes in serum protein analytes and whole blood RNA obtained over time will be summarized by treatment group. Associations between baseline levels and changes from baseline in select markers and response to treatment will be explored. RNA analyses will be summarized in a separate technical report.

The biomarker analyses will characterize the effects of guselkumab to identify biomarkers relevant to treatment, and to determine if these biomarkers can predict response to guselkumab. Results of serum, whole blood analyses, stool, and mucosal biopsy analyses will be reported in separate technical reports.

### Pharmacokinetic/Pharmacodynamic Analyses

The relationship between serum guselkumab concentrations and efficacy measures will be analyzed graphically. If any visual trend is observed, a suitable population PK/PD model may be developed to describe the E-R relationship. Details will be provided in a population PK/PD analysis plan and results of the population PK/PD analysis will be presented in a separate technical report.

### Medical Resource Utilization and Health Economics Analyses

Medical resource utilization and health economics, including work productivity, will be summarized by treatment group.

1-13. (canceled)

14. A method of treating Crohn's disease in a patient considered a biologic therapy failure or intolerance for Crohn's disease (Bio-Failure), a conventional therapy failure or intolerance for Crohn's disease (Con-Failure), or a TNF-inhibitor failure or intolerance for Crohn's disease (TNF-Failure), comprising administering an antibody to IL-23 to the patient, wherein the antibody comprises a light chain variable region and a heavy chain variable region, said light chain variable region comprising:
a complementarity determining region light chain 1 (CDRL1) amino acid sequence of SEQ ID NO:50;
a CDRL2 amino acid sequence of SEQ ID NO:56; and
a CDRL3 amino acid sequence of SEQ ID NO:73,
said heavy chain variable region comprising:
a complementarity determining region heavy chain 1 (CDRH1) amino acid sequence of SEQ ID NO:5;
a CDRH2 amino acid sequence of SEQ ID NO:20; and
a CDRH3 amino acid sequence of SEQ ID NO:44.

15. The method of claim 14, wherein the antibody is administered in an initial intravenous dose, an intravenous dose 4 weeks after initial treatment, an intravenous dose 8 weeks after initial treatment and a subcutaneous dose every 4 or 8 weeks after the dose at 8 weeks.

16. The method of claim 15, wherein the intravenous dose is selected from the group consisting of 16 mg/kg of the patient, 1200 mg, 600 mg and 200 mg.

17. The method of claim 16, wherein the subcutaneous dose is 100 mg or 200 mg.

18. The method of claim 17, wherein the intravenous dose is 1200 mg and the subcutaneous dose is 200 mg every 4 weeks.

19. The method of claim 17, wherein the intravenous dose is 600 mg and the subcutaneous dose is 200 mg every 4 weeks.

20. The method of claim 17, wherein the intravenous dose is 200 mg and the subcutaneous dose is 200 mg every 4 weeks.

21. The method of claim 17, wherein the intravenous dose is 200 mg and the subcutaneous dose is 100 mg every 8 weeks.

22. The method of claim 17, wherein the intravenous dose is 200 mg and the subcutaneous dose is 200 mg every 8 weeks.

23. The method of claim 14, wherein the patient is a responder to the antibody and is identified as meeting at least two clinical endpoints selected from the group consisting of:
(i) Change from Baseline in the Crohn's Disease Activity Index (CDAI) Score at Week 12;
(ii) Clinical remission at Week 12, defined as CDAI less than (<) 150 points;
(iii) Clinical response at Week 12, defined as greater than or equal to (>=) 100-point reduction from baseline in CDAI score or CDAI score<150;
(iv) Patient-Reported Outcome (PRO)-2 Remission at Week 12 defined based on average daily stool frequency (SF) and average daily abdominal pain (AP) score;
(v) Clinical-Biomarker Response at Week 12 defined using clinical response based on the CDAI score and reduction from baseline in C-reactive protein (CRP) or fecal calprotectin;
(vi) Endoscopic Response at Week 12 measured by the Simple Endoscopic Score for Crohn's Disease (SES-CD) is based on the evaluation of 4 endoscopic components across 5 ileocolonic segments, with a total score ranging from 0 to 56;
(vii) Clinical remission at Week 48 defined as CDAI score<150;
(viii) Durable Clinical Remission at Week 48 defined as CDAI<150 for most of all visits between Week 12 and Week 48;
(ix) Corticosteroid-Free Clinical Remission at Week 48 defined as CDAI score<150 at Week 48 and not receiving corticosteroids at Week 48;
(x) PRO-2 remission at Week 48 defined based on average daily stool frequency (SF) and average daily abdominal pain (AP) score;
(xi) Fatigue response at Week 12 based on the Patient-Reported Outcomes Measurement Information System (PROMIS) Fatigue Short Form 7a; and
(xii) Endoscopic response at Week 48 measured by the Simple Endoscopic Score for Crohn's Disease (SES-CD).

24. The method of claim 23, wherein the clinical endpoints are measured 8, 12, 16, 20, 28, 32, 36, 40, 44 and/or 48 weeks after initial treatment.

25. The method of claim 14, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the composition; wherein the composition comprises a diluent of water at standard state.

26. The method of claim 14, further comprising administering to the patient one or more additional drugs used to treat Crohn's disease.

27. The method of claim 26, wherein the additional drug is selected from the group consisting of: immunosuppressive agents, non-steroidal anti-inflammatory drugs (NSAIDs), methotrexate (MTX), anti-B-cell surface marker antibodies, anti-CD20 antibodies, rituximab, TNF-inhibitors, corticosteroids, and co-stimulatory modifiers.

28. The method of claim 14, wherein the antibody comprises a light chain variable region of the amino acid sequence of SEQ ID NO: 116 and a heavy chain variable region of the amino acid sequence of SEQ ID NO: 106.

29. The method of claim 14, wherein the antibody is guselkumab.
